(19) United States
(12) Patent Application Publication
(10) Pub. No.: US 2018/0303604 A1
(43) Pub. Date: Oct. 25, 2018

(54) Tissue Fixation Systems, Delivery Tools, and Associated Methods and Kits

(71) Applicants: Jeffry Melsheimer, Springville, IN (US); Gordon Lents, Louisville, KY (US)

(72) Inventors: Jeffry Melsheimer, Springville, IN (US); Gordon Lents, Louisville, KY (US)

(21) Appl. No.: 15/961,116

(22) Filed: Apr. 24, 2018

Related U.S. Application Data
(60) Provisional application No. 62/489,567, filed on Apr. 25, 2017.

(51) Int. Cl. A61F 2/20

(52) U.S. Cl. CPC A61F 2/20; A61F 2250/0067; A61F 2240/001; A61F 2220/0016

(57) ABSTRACT

Tissue fixation systems and associated methods and kits are described herein. An example embodiment of a tissue fixation system includes a locking screw and a tissue manipulator. The locking screw has a locking screw lengthwise axis and a locking screw main body that defines a locking screw exterior thread that extends in a first direction around the locking screw lengthwise axis and a locking screw interior thread that extends in a second direction around the locking screw lengthwise axis that is the same as the first direction. The tissue manipulator has a tissue manipulator lengthwise axis, a shaft, and a coil. The shaft has a shaft main body that defines a shaft exterior thread that extends in a third direction around the tissue manipulator lengthwise axis that is the same as the first direction. The coil extends from the shaft in a fourth direction around the tissue manipulator lengthwise axis that is different than the first direction.

### RELATED APPLICATIONS

This application claims the benefit of U.S. Provisional Application No. 62/489,567, filed on Apr. 25, 2017. The disclosure of this related application is hereby incorporated into this disclosure by reference.

### FIELD

The disclosure relates generally to the field of medical devices. More particularly, the disclosure relates to tissue fixation systems, delivery tools, methods of forming a tissue manipulator, kits that include a tissue fixation system, and methods that use a tissue fixation system.

### BACKGROUND

Vocal cord paralysis can impair breathing, swallowing, and phonation. Various procedures have been developed to reposition immobile vocal cords into a phonatory position to improve phonation, swallowing, and breathing in patients inflicted with vocal cord paralysis. For example, some procedures reposition the vocal cord via arytenoid adduction or arytenopexy coupled with cricothyroid approximation. However, these procedures have not gained wide acceptance because of the complexity and invasiveness of the deep neck dissection required to establish access to the arytenoid(s). Another invasive procedure, thyroplasty, requires removal of a portion of the thyroid cartilage and implantation of a piece of material that acts as a shim to push the vocal fold to the midline. Once placed, though, the shim usually is not adjustable and the procedure itself is difficult to reverse. Injection laryngoplasty, which involves the injection of a material into the vocal folds to achieve a desired repositioning, offers a less invasive option. Unfortunately, though, multiple injections may be required to achieve a desired result and, for some patients, the results can be temporary.

Therefore, a need exists for new and useful approaches to vocal cord treatment.

### SUMMARY OF SELECTED EXAMPLE EMBODIMENTS

Various tissue fixation systems, delivery tools, methods of forming a tissue manipulator, kits that include a tissue fixation system, and methods that use a tissue fixation system are described herein.

An example tissue fixation system comprises a locking screw and a tissue manipulator. The locking screw has a locking screw lengthwise axis, a locking screw first end, a locking screw second end, a locking screw axial length that extends from the locking screw first end to the locking screw second end, and a locking screw main body. The locking screw main body defines a locking screw passageway that extends from the locking screw first end to the locking screw second end, a locking screw exterior thread, and a locking screw interior thread. The locking screw exterior thread extends along a portion of the locking screw axial length and has a locking screw exterior thread first end and a locking screw exterior thread second end. The locking screw interior thread is disposed within the locking screw passageway and extends along a portion of the locking screw axial length and has a locking screw interior thread first end and a locking screw interior thread second end. The locking screw exterior thread extends from the locking screw exterior thread first end to the locking screw exterior thread second end and around the locking screw lengthwise axis in a first direction. The locking screw interior thread extends from the locking screw interior thread first end to the locking screw interior thread second end and around the locking screw lengthwise axis in a second direction that is the same as the first direction. The tissue manipulator has a tissue manipulator first end, a tissue manipulator second end, a tissue manipulator axial length that extends from the tissue manipulator first end to the tissue manipulator second end, a shaft, and a coil. The shaft is partially disposed through the locking screw passageway and has a shaft lengthwise axis, a shaft first end, a shaft second end, a shaft exterior surface, and a shaft main body that extends from the shaft first end to the shaft second end and defines a shaft exterior thread that extends along a portion of the tissue manipulator axial length. The shaft exterior thread is sized and configured to mate with the locking screw interior thread and has a shaft exterior thread first end and a shaft exterior thread second end. The shaft exterior thread extends from the shaft exterior thread first end to the shaft exterior thread second end and around the tissue manipulator lengthwise axis in a third direction that is the same as the first direction. The coil extends from the shaft second end to the tissue manipulator second end and around the tissue manipulator lengthwise axis in a fourth direction that is different than the first direction.

Another example tissue fixation system comprises a locking screw and a tissue manipulator. The locking screw has a locking screw lengthwise axis, a locking screw first end, a locking screw second end, a locking screw axial length that extends from the locking screw first end to the locking screw second end, and a locking screw main body. The locking screw main body defines a locking screw passageway that extends from the locking screw first end to the locking screw second end, a locking screw exterior thread, a locking screw interior thread, and a recess. The locking screw exterior thread extends along a portion of the locking screw axial length and has a locking screw exterior thread first end and a locking screw exterior thread second end. The locking screw interior thread is disposed within the locking screw passageway and extends along a portion of the locking screw axial length and has a locking screw interior thread first end and a locking screw interior thread second end. The locking screw exterior thread extends from the locking screw exterior thread first end to the locking screw exterior thread second end and around the locking screw lengthwise axis in a first direction. The locking screw interior thread extends from the locking screw interior thread first end to the locking screw interior thread second end and around the locking screw lengthwise axis in a second direction that is the same as the first direction. The recess extends from the locking screw first end toward the locking screw second end and is in communication with the locking screw passageway. The tissue manipulator has a tissue manipulator first end, a tissue manipulator second end, a tissue manipulator axial length that extends from the tissue manipulator first end to the tissue manipulator second end, a shaft, and a coil. The shaft is partially disposed through the locking screw passageway and has a shaft lengthwise axis, a shaft first end, a shaft second end, a shaft exterior surface, and a shaft main body that extends from the shaft first end to the shaft second end and defines a shaft exterior thread that extends along a portion of the tissue manipulator axial length. The shaft exterior thread is sized and configured to mate with the locking screw interior thread and has a shaft exterior thread first end and a shaft exterior thread second end. The shaft exterior thread extends from the shaft exterior thread first end to the shaft exterior thread second end and around the tissue manipulator lengthwise axis in a third direction that is the same as the first direction. The coil has a plurality of turns and extends from the shaft second end to the tissue manipulator second end and around the tissue manipulator lengthwise axis in a fourth direction that is different than the first direction.

Another example tissue fixation system comprises a locking screw and a tissue manipulator. The locking screw has a locking screw lengthwise axis, a locking screw first end, a locking screw second end, a locking screw axial length that extends from the locking screw first end to the locking screw second end, and a locking screw main body. The locking screw main body defines a locking screw passageway that extends from the locking screw first end to the locking screw second end, a locking screw exterior thread, a locking screw interior thread, and a recess. The locking screw exterior thread extends along a portion of the locking screw axial length and has a locking screw exterior thread first end and a locking screw exterior thread second end. The locking screw interior thread is disposed within the locking screw passageway and extends along a portion of the locking screw axial length and has a locking screw interior thread first end and a locking screw interior thread second end. The locking screw exterior thread extends from the locking screw exterior thread first end to the locking screw exterior thread second end and around the locking screw lengthwise axis in a counterclockwise direction. The locking screw interior thread extends from the locking screw interior thread first end to the locking screw interior thread second end and around the locking screw lengthwise axis in a second direction that is the same as the first direction. The recess extends from the locking screw first end toward the locking screw second end and is in communication with the locking screw passageway. The tissue manipulator has a tissue manipulator first end, a tissue manipulator second end, a tissue manipulator axial length that extends from the tissue manipulator first end to the tissue manipulator second end, a shaft, and a coil. The shaft is partially disposed through the locking screw passageway and has a shaft lengthwise axis, a shaft first end, a shaft second end, a shaft exterior surface, and a shaft main body that extends from the shaft first end to the shaft second end and defines a shaft exterior thread that extends along a portion of the tissue manipulator axial length. The shaft exterior thread is sized and configured to mate with the locking screw interior thread and has a shaft exterior thread first end, a shaft exterior thread second end, and a shaft exterior thread pitch. The shaft exterior thread extends from the shaft exterior thread first end to the shaft exterior thread second end and around the tissue manipulator lengthwise axis in a third direction that is the same as the first direction. The coil has a plurality of turns and extends from the shaft second end to the tissue manipulator second end and around the tissue manipulator lengthwise axis in a fourth direction that is different than the first direction. The coil has a coil pitch that is different than the shaft exterior thread pitch.

An example delivery tool comprises a delivery tool lengthwise axis, a delivery tool first end, a delivery tool second end, a delivery tool axial length, and a delivery tool main body that defines an elongate member, a plurality of projections, and a delivery tool passageway that extends from the delivery tool first end to the delivery tool second end. The elongate member has an elongate member first end and an elongate member second end. Each projection of the plurality of projections extends from the elongate member second end to the delivery tool second end and is sized and configured to mate with a recess of a locking screw such that the delivery tool can be used to advance and withdraw the locking screw into and out another feature, element, or component, such as the thyroid cartilage of a patient.

Another example delivery tool comprises a delivery tool lengthwise axis, a delivery tool first end, a delivery tool second end, a delivery tool axial length, and a delivery tool main body that defines an elongate member, a projection, and a delivery tool passageway that extends from the delivery tool first end to the delivery tool second end. The elongate member has an elongate member first end and an elongate member second end. The projection extends from the elongate member second end to the delivery tool second end and is sized and configured to mate with a recess of a locking screw such that the delivery tool can be used to advance and withdraw the locking screw into and out another feature, element, or component, such as the thyroid cartilage of a patient.

An example method of manufacturing a tissue manipulator comprises the steps of: selecting a wire member to form a tissue manipulator, the wire member has a wire member first end and a wire member second end; forming an exterior thread along a portion of the axial length of the wire member; reducing the outside diameter of the wire member along a portion of its axial length that extends from the wire member second end toward the wire member first end; forming one or more turns along the portion of the axial length of the wire member that extends from the wire member second end toward the wire member first end to form a coil; trimming the wire member to a desired length; forming a sharpened tip on the wire member second end such that the wire member second end is sharp relative to the wire member first end.

Another method of manufacturing a tissue manipulator comprises the steps of: selecting a cannula; forming an exterior thread along a portion of the axial length of the cannula; selecting a wire member, the wire member having a wire member first end and a wire member second end; forming one or more turns along a portion of the axial length of the wire member that extends from the wire member second end toward the wire member first end to form a coil; inserting the wire member into a lumen defined by the cannula; attaching the wire member to the cannula; trimming the wire member to a desired length; forming a sharpened tip on the wire member second end such that the wire member second end is sharp relative to the wire member first end.

An example kit that includes a tissue fixation system comprises a first tissue fixation system according to an embodiment; a second tissue fixation system according to an embodiment; a first delivery tool according to an embodiment; a second delivery tool according to an embodiment; and instructions for use.

An example method of treatment using a tissue fixation system comprises the steps of: creating an opening in the body over the vocal cord intended to be treated to access the thyroid cartilage; introducing a needle and an obturator through the opening and the thyroid cartilage and into the arytenoid muscle; withdrawing the obturator while maintaining the position of the needle; passing a tissue manipulator through the needle until it contacts the arytenoid muscle; applying torque on the tissue manipulator while applying an axial force toward the arytenoid muscle such that the tissue manipulator rotates and engages the arytenoid muscle; confirming engagement of the tissue manipulator with the arytenoid muscle; withdrawing the needle from the tissue and thyroid cartilage while maintaining position of the tissue manipulator; advancing a locking screw that has a locking screw first end, a locking screw second end, and a locking screw main body that defines a locking screw passageway over the tissue manipulator such that a portion of the tissue manipulator is disposed within the locking screw passageway; applying torque on the locking screw while maintaining the position of the tissue manipulator such that the locking screw rotates relative to the tissue manipulator and engages the thyroid cartilage; continuing the application of torque on the locking screw while maintaining the position of the tissue manipulator until the locking screw first end is substantially flush with a surface of the thyroid cartilage; asking the patient to phonate to evaluate the position of the vocal cord and to determine whether adjustments are needed; trimming the tissue manipulator; closing the opening made in the body.

Additional understanding of the example tissue fixation systems, methods of forming a tissue manipulator, kits that include a tissue fixation system, and methods that use a tissue fixation system can be obtained by review of the detailed description, below, and the appended drawings.

### BRIEF DESCRIPTION OF THE DRAWINGS

FIG. 1 is a perspective view of a first example tissue fixation system.

FIG. 2 is a perspective view of the locking screw of the tissue fixation system illustrated in FIG. 1.

FIG. 3 is a sectional view of the locking screw illustrated in FIG. 2 taken along line 3-3.

FIG. 4 is a perspective view of the tissue manipulator of the tissue fixation system illustrated in FIG. 1.

FIG. 5 is a perspective view of another example tissue fixation system.

FIG. 6 is a perspective view of the locking screw of the tissue fixation system illustrated in FIG. 5.

FIG. 7 is a sectional view of the locking screw illustrated in FIG. 6 taken along line 7-7.

FIG. 8 is a perspective view of the tissue manipulator of the tissue fixation system illustrated in FIG. 5.

FIG. 9 is a perspective view of an example delivery tool used to implant a tissue fixation system.

FIG. 10 is an end view of the second end of the delivery tool illustrated in FIG. 9.

FIG. 11 is a perspective view of another example delivery tool used to implant a tissue fixation system.

FIG. 12 is an end view of the second end of the delivery tool illustrated in FIG. 11.

FIG. 13 is a schematic illustration of an example method of manufacturing a tissue manipulator.

FIG. 14 is a schematic illustration of another example method of manufacturing a tissue manipulator.

FIG. 15 illustrates an example kit that includes a tissue fixation system.

FIG. 16 is a schematic illustration of an example method of treatment using a tissue fixation system.

FIG. 17 illustrates a superior view of the thyroid cartilage and the arytenoid muscle with a needle and obturator disposed through the thyroid cartilage and partially disposed within the arytenoid muscle.

FIG. 18 illustrates a superior view of the thyroid cartilage and the arytenoid muscle with a needle disposed through the thyroid cartilage and partially disposed within the arytenoid muscle.

FIG. 19 illustrates a superior view of the thyroid cartilage and the arytenoid muscle with a needle and a tissue manipulator disposed through the thyroid cartilage and partially disposed within the arytenoid muscle.

FIG. 20 illustrates a superior view of the thyroid cartilage and the arytenoid muscle with a needle and a tissue manipulator disposed through the thyroid cartilage and partially disposed within the arytenoid muscle. The tissue manipulator has been advanced into the arytenoid muscle.

FIG. 21 illustrates a superior view of the thyroid cartilage and the arytenoid muscle with a tissue manipulator disposed through the thyroid cartilage and partially disposed within the arytenoid muscle.

FIG. 22 illustrates a superior view of the thyroid cartilage and the arytenoid muscle with a tissue manipulator disposed through the thyroid cartilage and partially disposed within the arytenoid muscle. A locking screw has been partially advanced over the tissue manipulator and is being advanced toward the thyroid cartilage.

FIG. 23 illustrates a superior view of the thyroid cartilage and the arytenoid muscle with a tissue manipulator disposed through the thyroid cartilage and partially disposed within the arytenoid muscle. A locking screw has been partially advanced over the tissue manipulator and is being advanced into the thyroid cartilage using a delivery tool.

FIG. 24 illustrates a superior view of the thyroid cartilage and the arytenoid muscle with a tissue manipulator disposed through the thyroid cartilage and partially disposed within the arytenoid muscle. The locking screw has been partially advanced over the tissue manipulator and is disposed within the thyroid cartilage such that the locking screw first end is substantially flush with a surface of the thyroid cartilage.

FIG. 25 illustrates a superior view of the thyroid cartilage and the arytenoid muscle with a locking screw attached to the thyroid cartilage and a tissue manipulator disposed through the locking screw passageway and partially disposed within the arytenoid muscle.

### DETAILED DESCRIPTION

The following detailed description and the appended drawings describe and illustrate various example embodiments of tissue fixation systems, delivery tools, methods of forming a tissue manipulator, kits that include a tissue fixation system, and methods that use a tissue fixation system. The description and illustration of these examples are provided to enable one skilled in the art to make and use a tissue fixation system, delivery tool, to practice a method of manufacturing a tissue manipulator, to practice a method of treatment using a tissue fixation system, and to make a kit that includes a tissue fixation system. They are not intended to limit the scope of the claims in any manner.

As used herein, the term “engaged” and grammatically related terms refer to a first element or feature being associated with, interconnected with, disposed in, or attached to a second element or feature such that the first element or feature can alter the position of the second element or feature when an outside force is applied on a portion of the first element or feature.

As used herein, the term “fixation” refers to the ability of a first device, element, or feature to maintain a second device, element, or feature in a particular position.

As used herein, the phrase “arytenoid muscle” refers to the arytenoid muscle, the thyroarytenoid muscle, the cricoarytenoid muscle, and the arytenoid muscular process.

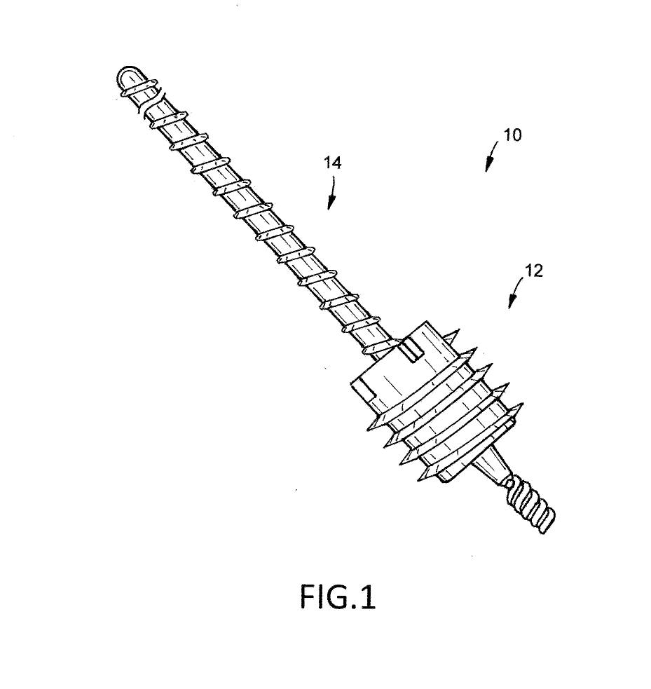

FIGS. 1, 2, 3, and 4 illustrate a first example tissue fixation system 10. The tissue fixation system 10 includes a locking screw 12 and a tissue manipulator 14. In the illustrated embodiment, the tissue manipulator 14 is partially disposed through the locking screw 12 and the locking screw 12 and tissue manipulator 14 are rotatable relative to one another, as described in more detail herein.

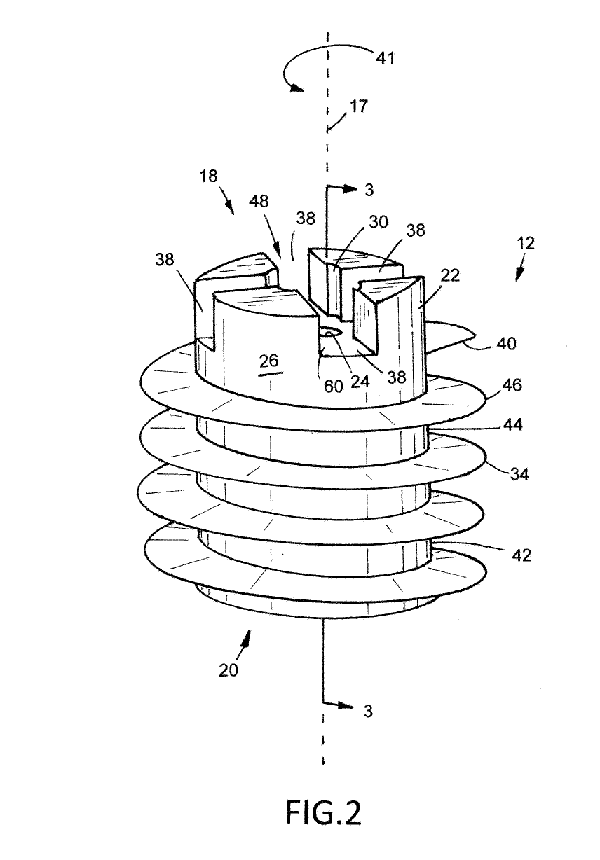

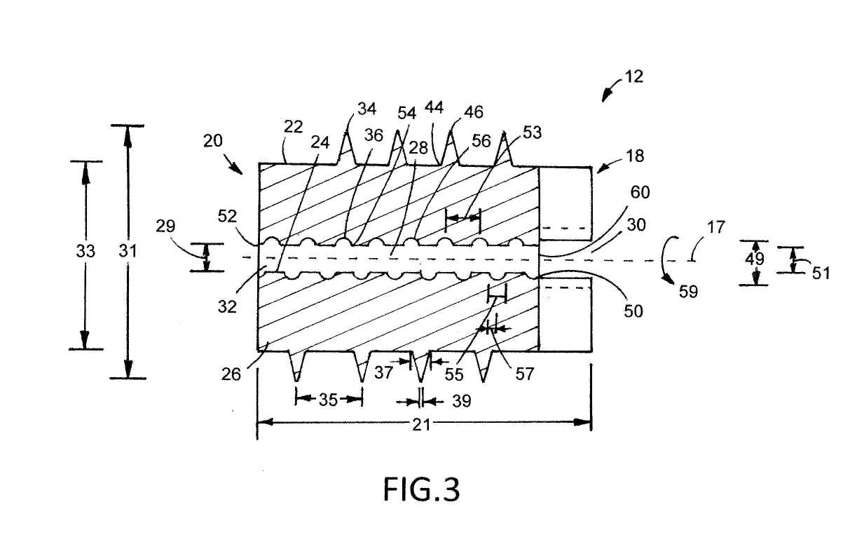

As shown in FIGS. 2 and 3, the locking screw 12 has a locking screw lengthwise axis 17, a locking screw first end 18, a locking screw second end 20, a locking screw axial length 21, a locking screw outer surface 22, a locking screw inner surface 24, and a locking screw main body 26. The locking screw axial length 21 extends from the locking screw first end 18 to the locking screw second end 20. The locking screw main body 26 defines a locking screw passageway 28, a locking screw first opening 30, a locking screw second opening 32, a locking screw exterior thread 34, a locking screw interior thread 36, and a plurality of recesses 38. The locking screw passageway 28 extends from the locking screw first opening 30 defined on the locking screw first end 18 to the locking screw second opening 32 defined on the locking screw second end 20. The locking screw passageway 28 has an inside diameter 29.

The locking screw exterior thread 34 extends from the locking screw outer surface 22, away from the locking screw lengthwise axis 17, and along a portion of the locking screw axial length 21. The locking screw exterior thread 34 has a locking screw exterior thread first end 40, a locking screw exterior thread second end 42, a locking screw exterior thread root 44, a locking screw exterior thread crest 46, a locking screw exterior thread major diameter 31, a locking screw exterior thread minor diameter 33, a locking screw exterior thread pitch 35, a locking screw exterior thread first thickness 37, and a locking screw exterior thread second thickness 39. In the illustrated embodiment, the locking screw exterior thread first end 40 is disposed between the locking screw first end 18 and the locking screw second end 20 and the locking screw exterior thread second end 42 is disposed between the locking screw exterior thread first end 40 and the locking screw second end 20. The locking screw exterior thread 34 extends from the locking screw exterior thread first end 40 to the locking screw exterior thread second end 42 and around the locking screw lengthwise axis 17 in a first direction 41. In the illustrated embodiment, the first direction 41 is counterclockwise.

In the illustrated embodiment, the locking screw exterior thread first thickness 37 is disposed adjacent the locking screw outer surface 22 and the locking screw exterior thread second thickness 39 is disposed between the locking screw exterior thread first thickness 37 and the locking screw exterior thread crest 46. In the illustrated embodiment, the locking screw exterior thread first thickness 37 is greater than the locking screw exterior thread second thickness 39 and the locking screw exterior thread thickness tapers from the locking screw exterior thread root 44 to the locking screw exterior thread crest 46.

While the locking screw exterior thread first end 40 has been illustrated as disposed between the locking screw first end 18 and the locking screw second end 20 and the locking screw exterior thread second end 42 has been illustrated as disposed between the locking screw exterior thread first end 40 and the locking screw second end 20, a locking screw exterior thread first end and a locking screw exterior thread second end can be positioned at any suitable location on a locking screw. Selection of a suitable location to position a locking screw exterior thread first end and a locking screw exterior thread second end can be based on various considerations, such as the structural arrangement of a locking screw and/or a tissue manipulator that is intended to be passed through a locking screw. Example locations considered suitable to position a locking screw exterior thread first end and/or a locking screw exterior thread second end include positioning a locking screw exterior thread first end at the locking screw first end or between the locking screw first end and the locking screw second end, positioning a locking screw exterior thread second end at the locking screw second end or between the locking screw first end and the locking screw second end, and/or any other location considered suitable for a particular embodiment.

The locking screw interior thread 36 is disposed within the locking screw passageway 28 and extends along a portion of the locking screw axial length 21. The locking screw interior thread 36 has a locking screw interior thread first end 50, a locking screw interior thread second end 52, a locking screw interior thread root 54, a locking screw interior thread crest 56, a locking screw interior thread major diameter 49, a locking screw interior thread minor diameter 51, a locking screw interior thread pitch 53, a locking screw interior thread first thickness 55, and a locking screw interior thread second thickness 57. In the illustrated embodiment, the locking screw interior thread first end 50 is disposed between the locking screw first end 18 and the locking screw second end 20 and the locking screw interior thread second end 52 is disposed at the locking screw second end 20. The locking screw interior thread 36 extends from the locking screw interior thread first end 50 to the locking screw interior thread second end 52 and around the locking screw lengthwise axis 17 in a second direction 59. In the illustrated embodiment, the second direction 59 is the same as the first direction 41 and is counterclockwise.

In the illustrated embodiment, the locking screw interior thread first thickness 55 is disposed adjacent the locking screw inner surface 24 and the locking screw interior thread second thickness 57 is disposed between the locking screw interior thread first thickness 55 and the locking screw interior thread crest 56. In the illustrated embodiment, the locking screw interior thread first thickness 55 is greater than the locking screw interior thread second thickness 57 and the locking screw interior thread thickness tapers from the locking screw interior thread first thickness 55 to the locking screw interior thread second thickness 57.

Each recess of the plurality of recesses 38 extends from the locking screw first end 18 toward the locking screw second end 20 to a recess base 60, from the locking screw outer surface 22 to the locking screw interior surface 24, and is in communication with the locking screw passageway 28. Each recess of the plurality of recesses 38 is sized and configured to receive a portion of a tissue manipulator, such as tissue manipulator 14, and/or a tool that is sized and configured to trim a portion of a tissue manipulator. In addition, each recess of the plurality of recesses 38 cooperatively defines an opening 48 that is sized and configured to receive a portion of a delivery tool, such as delivery tool 400 described in more detail herein, that is configured to engage the locking screw 12 and advance or withdraw the locking screw 12 into and out of another feature, element, or component, such as the thyroid cartilage of a patient.

A locking screw included in a tissue fixation system can have any suitable structural arrangement and selection of a suitable structural arrangement for a locking screw can be based on various considerations, including the structural arrangement of an implant site and/or the structural arrangement of a tissue manipulator included in the tissue fixation system. For example, a locking screw exterior thread and a locking screw interior thread can have any suitable major diameter, minor diameter, thickness, and pitch. Examples of suitable locking screw exterior thread major diameters include major diameters that are between about 0.1 inches and about 0.3 inches, between about 0.15 inches and about 0.25 inches, equal to, less than, or greater than about 0.242 inches, and any other major diameter considered suitable for a particular embodiment. In the illustrated embodiment, the locking screw exterior thread major diameter 31 is equal to about 0.242 inches. Examples of suitable locking screw exterior thread minor diameters include minor diameters that are between about 0.1 inches and about 0.3 inches, between about 0.125 inches and about 0.2 inches, equal to, less than, or greater than about 0.156 inches, and any other minor diameter considered suitable for a particular embodiment. In the illustrated embodiment, the minor diameter 33 is equal to about 0.156 inches. Examples of suitable locking screw exterior thread pitches include pitches that are between about 5 threads per inch and about 15 threads per inch, between about 7 threads per inch and about 13 threads per inch, equal to, less than, or greater than about 10 threads per inch, and any other pitch considered suitable for a particular embodiment. In the illustrated embodiment, the locking screw exterior thread pitch 35 is equal to about 10 threads per inch. Examples of suitable passageway inside diameters include inside diameters between about 0.02 inches and about 0.06 inches, between about 0.03 inches and about 0.05 inches, equal to, less than, or greater than about 0.047 inches, and any other inside diameter considered suitable for a particular embodiment. In the illustrated embodiment, the passageway inside diameter 29 is equal to about 0.047 inches. Examples of suitable locking screw lengths include lengths that are equal to, less than, greater than, or about 0.5 inches, about 0.5 inches, and any other length considered suitable for a particular embodiment.

Examples of suitable locking screw interior thread major diameters include major diameters that are between about 0.04 inches and about 0.08 inches, between about 0.05 inches and about 0.07 inches, equal to, less than, or greater than about 0.06 inches, and any other major diameter considered suitable for a particular embodiment. In the illustrated embodiment, the locking screw interior thread major diameter 49 is equal to about 0.06 inches. Examples of suitable locking screw interior thread minor diameters include minor diameters that are between about 0.03 inches and about 0.06 inches, between about 0.04 inches and about 0.05 inches, equal to, less than, or greater than about 0.047 inches, and any other minor diameter considered suitable for a particular embodiment. In the illustrated embodiment, the minor diameter 51 is equal to about 0.047 inches. Examples of suitable locking screw interior thread pitches include pitches that are between about 5 threads per inch and about 15 threads per inch, between about 7 threads per inch and about 13 threads per inch, equal to, less than, or greater than about 10 threads per inch, and any other pitch considered suitable for a particular embodiment. In the illustrated embodiment, the locking screw interior thread pitch 53 is equal to the locking screw exterior thread pitch 35 and is about 10 threads per inch. However, in alternative embodiments, the locking screw interior thread pitch can be different than the locking screw exterior thread pitch.

While the plurality of recesses 38 have been illustrated as cooperatively defining an opening 48 that is sized and configured to receive a portion of a delivery tool, a locking screw can define any suitable number of recesses having any suitable shape that is sized and configured to receive a portion of a delivery tool. Selection of a suitable number of recesses, and of a suitable shape for a recess or a plurality of recesses, can be based on various considerations, including the structural arrangement of an implant site and/or the structural arrangement of a tissue manipulator included in the tissue fixation system. Examples of numbers of recesses considered suitable to include on a locking screw include one, at least one, two, a plurality, three, four, five, six, seven, eight, and any other number considered suitable for a particular embodiment. Example structural arrangements considered suitable for a recess, or a plurality of recesses, to define include structural arrangements that define a slotted recess, a Phillips recess, a Frearson recess, an Allen recess (e.g., hexagonal prism), and any other structural arrangement considered suitable for a particular embodiment.

While the locking screw interior thread first end 50 has been illustrated as disposed between the locking screw first end 18 and the locking screw second end 20 and the locking screw interior thread second end 52 has been illustrated as disposed at the locking screw second end 20, a locking screw interior thread first end and a locking screw interior thread second end can be positioned at any suitable location along a locking screw. Selection of a suitable location to position a locking screw interior thread first end and a locking screw interior thread second end can be based on various considerations, such as the structural arrangement of a locking screw and/or a tissue manipulator that is intended to be passed through a locking screw. Example locations considered suitable to position a locking screw interior thread first end and/or a locking screw interior thread second end include positioning a locking screw interior thread first end at the locking screw first end or between the locking screw first end and the locking screw second end, positioning a locking screw interior thread second end at the locking screw second end or between the locking screw first end and the locking screw second end, and/or any other location considered suitable for a particular embodiment.

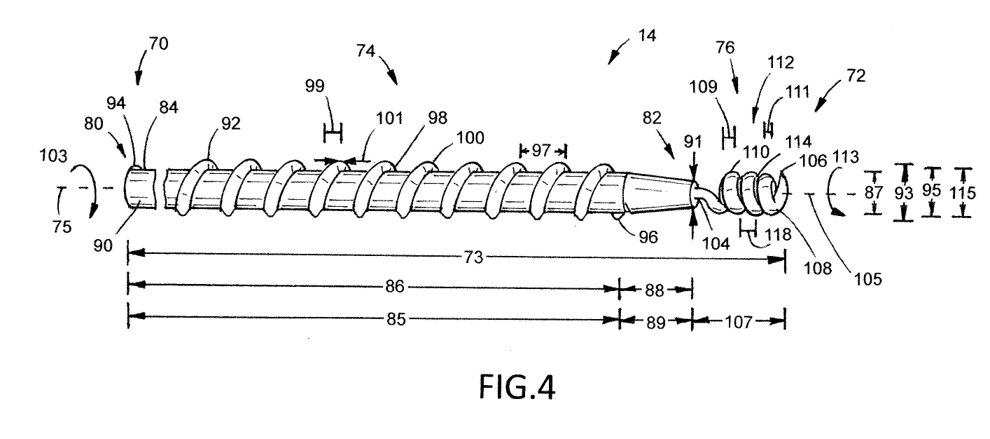

As shown in FIG. 4, the tissue manipulator 14 has a tissue manipulator first end 70, a tissue manipulator second end 72, a tissue manipulator axial length 73, a shaft 74, and a coil 76. The tissue manipulator axial length 73 extends from the tissue manipulator first end 70 to the tissue manipulator second end 72.

In the illustrated embodiment, the shaft 72 has a shaft lengthwise axis 75, a shaft first end 80, a shaft second end 82, a shaft exterior surface 84, a shaft first portion 86, a shaft second portion 88, and a shaft main body 90. The shaft main body 90 extends from the shaft first end 80 to the shaft second end 82 and defines a shaft exterior thread 92. The shaft lengthwise axis 75 extends through the shaft 72 from the shaft first end 80 to the shaft second end 82. In the illustrated embodiment, the shaft first portion 86 extends from the shaft first end 80 toward the shaft second end 82 and has a shaft first portion length 85 and a shaft first portion outside diameter 87. The shaft second portion 88 extends from the shaft first portion 86 to the shaft second end 82 and has a shaft second portion length 89 and a shaft second portion outside diameter 91 disposed at the shaft second end 82. In the illustrated embodiment, the shaft first portion length 85 is greater than the shaft second portion length 89 and the shaft first portion outside diameter 87 is greater than the shaft second portion outside diameter 91. In the illustrated embodiment, the outside diameter of the shaft 72 tapers from the shaft first portion 86 to the shaft second end 82 along the shaft second portion 88.

The shaft exterior thread 92 extends along a portion of the tissue manipulator axial length 73 and is sized and configured to mate with the locking screw interior thread 36. In the illustrated embodiment, the shaft exterior thread 92 extends from the shaft exterior surface 84, away from the shaft lengthwise axis 75, and from the shaft first end 80 to the shaft second end 82. The shaft exterior thread 92 has a shaft exterior thread first end 94, a shaft exterior thread second end 96, a shaft exterior thread root 98, a shaft exterior thread crest 100, a shaft exterior thread major diameter 93, a shaft exterior thread minor diameter 95, a shaft exterior thread pitch 97, a shaft exterior thread first thickness 99, and a shaft exterior thread second thickness 101. In the illustrated embodiment, the shaft exterior thread first end 94 is disposed at the shaft first end 80 and the shaft exterior thread second end 96 is disposed at the shaft second end 82. The shaft exterior thread 92 extends from the shaft exterior thread first end 94 to the shaft exterior thread second end 96 and around the shaft lengthwise axis 75 in a third direction 103 that is the same as the first direction 41. In the illustrated embodiment, the third direction 103 is counterclockwise.

In the illustrated embodiment, the shaft exterior thread pitch 97 is the same as the locking screw interior thread pith 53, the shaft exterior thread first thickness 99 is disposed adjacent the shaft exterior surface 84, and the shaft exterior thread second thickness 101 is disposed between the shaft exterior thread first thickness 99 and the shaft exterior thread crest 100. In the illustrated embodiment, the shaft exterior thread first thickness 99 is greater than the shaft exterior thread second thickness 101 and the shaft exterior thread thickness tapers from the shaft exterior thread root 98 to the shaft exterior thread crest 100.

While the shaft first portion length 85 has been illustrated as being greater than the shaft second portion length 89, a shaft first portion length and a shaft second portion length of a shaft can be any suitable length and selection of a suitable length can be based on various considerations, such as the structural arrangement of a locking screw through which a tissue manipulator is intended to be passed and/or the structural arrangement and/or material that forms the portion of the body to which the tissue manipulator is intended to be attached. For example, a shaft second portion can have a length that is equal to, or about, a coil main body length. Examples of lengths considered suitable for a shaft first portion length includes lengths equal to, greater than, less than, or about 4 inches, 5 inches, between about 3 inches and about 6 inches, and any other length considered suitable for a particular embodiment.

While the shaft first portion outside diameter 87 has been illustrated as being greater than the shaft second portion outside diameter 91, a shaft first portion outside diameter and a shaft second portion outside diameter of a shaft can be any suitable outside diameter and selection of a suitable outside diameter can be based on various considerations, such as the structural arrangement of a locking screw through which a tissue manipulator is intended to be passed and the structural arrangement and/or material that forms the portion of the body to which the tissue manipulator is intended to be attached.

While the shaft exterior thread first end 94 has been illustrated as disposed at the shaft first end 80 and the shaft exterior thread second end 96 has been illustrated as disposed at the shaft second end 82, a shaft exterior thread first end and a shaft exterior thread second end can be positioned at any suitable location along a shaft. Selection of a suitable location to position a shaft exterior thread first end and a shaft exterior thread second end can be based on various considerations, such as the structural arrangement of a locking screw through which a tissue manipulator is intended to be passed. Example locations considered suitable to position a shaft exterior thread first end and/or a shaft exterior thread second end include positioning a shaft exterior thread first end at the shaft first end or between the shaft first end and the shaft second end, positioning a shaft exterior thread second end at the shaft second end or between the shaft first end and the shaft second end, and/or any other location considered suitable for a particular embodiment.

While the locking screw exterior thread 34, locking screw interior thread 36, and shaft exterior thread 92 have been illustrated as having a particular structural configuration, a thread included on a locking screw or tissue manipulator can have any suitable thread configuration. Selection of a suitable thread configuration for a thread included on a locking screw or tissue manipulator can be based on various considerations, such as the material that forms a locking screw and/or a tissue manipulator and/or the treatment intended to be completed. Example thread configurations considered suitable for a locking screw exterior thread, a locking screw interior thread, a shaft exterior thread, or any other thread included on a locking screw and/or tissue manipulator include V threads, American National threads, unified threads, Whitworth threads, square threads, acme threads, buttress threads, knuckle threads, single start threads, double start threads, triple start threads, self-tapping threads, and any other thread configuration considered suitable for a particular embodiment. For example, alternative embodiments can include a locking screw exterior thread that extends from a locking screw exterior thread first end to a locking screw exterior thread second end and around a locking screw lengthwise axis in a clockwise direction, a locking screw interior thread that extends from a locking screw interior thread first end to a locking screw interior thread second end and around a locking screw lengthwise axis in a clockwise direction, a direction that is the same as the first direction, or a direction that is different than the first direction, a shaft exterior thread that extends from a shaft exterior thread first end to a shaft exterior thread second end and around a shaft lengthwise axis in a clockwise direction, a direction that is the same as the first direction, or a direction that is different than the first direction. In the illustrated embodiment, the locking screw exterior thread 34 is a self-tapping thread and is different than the locking screw interior thread 36.

In the illustrated embodiment, the coil 76 extends from the shaft second end 82 to the tissue manipulator second end 72. The coil 72 has a coil lengthwise axis 105, a coil first end 104, a coil second end 106, and a coil main body 108. The coil second end 106 is sharpened such that the coil second end 106 (e.g., tissue manipulator second end 72) is sharp relative to the tissue manipulator first end 70 and is configured to pierce tissue, such as cartilage, such as the thyroid cartilage, and/or muscle, such as the arytenoid muscle. While the coil second end 106 has been described as being sharpened, alternative embodiments can include a coil second end that is not sharpened, that is tapered, or that is atraumatic. The coil main body 108 has a coil main body length 107, a coil main body first outside diameter 109, a coil main body second outside diameter 111, and defines a plurality of coil turns 110 creating a helical coil 112. The coil lengthwise axis 105 is coaxial with the shaft lengthwise axis 75 and extends through the plurality of coil turns 110. The coil main body length 107 extends from the coil first end 104 to the coil second end 106 and is measured along the coil lengthwise axis 105. In the illustrated embodiment, the coil main body 108 extends from the coil first end 104 to the coil second end 106 and around the coil lengthwise axis 105 in a fourth direction 113 that is the different than the first direction 41. In the illustrated embodiment, the fourth direction 113 is clockwise. Alternative embodiments, however, can include a coil main body that extends from a coil first end to a coil second end and around a coil lengthwise axis in a fourth direction that is the same as the first direction.

In the illustrated embodiment, the plurality of coil turns 110 comprises 4 complete coil turns creating a 45 degree helical coil 112 that defines a space 114 between adjacent turns of the plurality of coil turns 110. The plurality of coil turns 110 has a maximum coil turn outside diameter 115 and the plurality of coil turns 110 have a coil pitch 118. In the illustrated embodiment, the length between each set of adjacent coil turns of the plurality of coils 110 is equal and the coil pitch 118 is the same as the shaft exterior thread pitch 97.

The coil main body first outside diameter 109 is less than the shaft first portion outside diameter 87 and is greater than the coil main body second outside diameter 111. In the illustrated embodiment, the outside diameter of the coil main body 108 tapers from the coil first end 104 to the coil second end 106. The coil outside diameter can taper at any suitable rate along the coil main body length 107 and the coil main body first outside diameter 109 and coil main body second outside diameter 111 can be any suitable outside diameter. Alternatively, the outside diameter of a coil main body can omit a taper such that it defines a constant outside diameter along its length. Selection of a suitable coil main body outside diameter can be based on various considerations, such as the structural arrangement of a locking screw through which a tissue manipulator is intended to be passed and the structural arrangement and/or material that forms the portion of the body to which the tissue manipulator is intended to be attached (e.g., thyroid cartilage). Example outside diameters considered suitable include outside diameters in which the coil main body first outside diameter is greater than, equal to, or less than the shaft second portion outside diameter, diameters in which the coil main body first outside diameter is greater than, equal to, or less than the coil main body second outside diameter, and any other outside diameter considered suitable for a particular embodiment. For example, a coil main body can have a coil main body first outside diameter that is equal to, less than, greater than, or about 0.014 inches, 0.021 inches, or between about 0.010 inches and about 0.025 inches.

While the coil 76 has been illustrated as having a coil lengthwise axis 105 that is coaxial with the shaft lengthwise axis 75, a plurality of coil turns 110 that extends in a clockwise direction, a maximum coil turn outside diameter 115, and defining a 45 degree helical coil 112 that defines a space 114 between adjacent turns of the coil 76, a coil can have any suitable structural arrangement. Selection of a suitable structural arrangement for a coil can be based on various considerations, such as the structural arrangement and/or material that forms the portion of the body to which a tissue manipulator is intended to be attached. For example, alternative embodiments can include a coil lengthwise axis that is not coaxial with a shaft lengthwise axis (e.g., a coil lengthwise axis is parallel with, or disposed at an angle to, a shaft lengthwise axis) and/or a coil that has a coil main body that extends from a coil first end to a coil second end and around a coil lengthwise axis in a counterclockwise direction, a direction that is the same as the first direction, or a direction that is different than the first direction. Examples of number of turns considered suitable to include on coil include a partial turn, one complete turn, at least one complete turn, two complete turns, three complete turns, four complete turns, five complete turns, six complete turns, seven complete turns, eight complete turns, between four and eight complete turns, more than eight complete turns, and any other number considered suitable for a particular embodiment. Examples of maximum coil turn outside diameters considered suitable for a coil include diameters equal to, about, less than, or greater than, one time, two times, three times, four times, or five times a shaft first portion outside diameter or a shaft second portion outside diameter, those that are equal to, greater than, less than, or about 0.042 inches, 0.063 inches, between about 0.035 inches and about 0.70 inches, and any other diameter considered suitable for a particular embodiment. Examples of types of coils that can be created by the coil main body of a coil include helical coils, conical coils, 45 degree helical coils, 45 degree conical coils, coils that have a rectangular, square, round, or elongated, cross-sectional shape, coils that have any suitable spacing between adjacent turns, coils that have any suitable angular arrangement, and any other coil considered suitable for a particular embodiment. Examples of suitable pitches for a coil include pitches that are greater than, equal to, or less than, the pitch of a locking screw exterior thread, a locking screw interior thread, and/or a shaft exterior thread, pitches that are different than the pitch of a locking screw exterior thread, a locking screw interior thread, and/or a shaft exterior thread, and any other configuration considered suitable for a particular embodiment. Example distances considered suitable between adjacent turns of a plurality of coil turns include distances that are equal to one another, that vary, that increase, or decrease, from the coil first end to the coil second end, and any other distance considered suitable for a particular embodiment.

While the outside diameter of the coil main body 108 has been illustrated as tapering from the coil first end 104 to the coil second end 106, the coil main body of a coil can taper along any suitable portion of a coil main body length. Selection of a suitable main body length to include a tapering diameter can be based on various considerations, such as the structural arrangement and/or material that forms the portion of the body to which a tissue manipulator is intended to be attached. For example, the outside diameter of a coil main body can taper from a first location disposed between the coil first end and the coil second end to the second end, or a second location disposed between the first location and the coil second end. Alternatively, the outside diameter of a coil main body can be constant along the coil main body length.

While the shaft 74 and coil 76 have been illustrated as being formed from a unitary piece of material, a shaft and coil can comprise separate components attached to one another using any suitable technique or method of attachment. Selection of a suitable technique or method of attachment to use between a shaft and a coil can be based on various considerations, such as the material(s) that forms the shaft and/or the coil. Example techniques and methods of attachment considered suitable between a shaft and a coil include welding, fusing, using adhesive, and any other technique or method considered suitable for a particular embodiment.

A locking screw 12 and tissue manipulator 14 of a tissue fixation system 10 can be formed of any suitable material and selection of a suitable material to form a locking screw and/or tissue manipulator according to a particular embodiment can be based on various considerations, including the material forming the portion of the body to which a locking screw and/or tissue manipulator is intended to be attached. Examples of materials considered suitable to form a locking screw and/or a tissue manipulator include rigid materials, semi-rigid materials, implant grade materials, implant grade rigid materials, implant grade semi-rigid materials, biocompatible materials, materials that can be made biocompatible, metals such as stainless steel, titanium, metal alloys, nickel-titanium alloy (e.g., Nitinol), thermoplastics, polymers, polyetheretherketone (PEEK), Pebax (Pebax is a registered trademark of Ato Chimie Corporation of Allee des Vosges, Courbevoie, France), nylon, polyethylene, high-density polyethylene (HDPE), high-performance polyethylene (HPPE), polyurethane, silicone, acrylonitrile butadiene styrene (ABS), polyoxymethylene (e.g., acetal), materials that have a round cross-sectional configuration, materials that have a rectangular, elongated, or square, cross-sectional configuration, materials that have a first cross-sectional configuration along a first portion of its length (e.g., round) and a second cross-sectional configuration along a second portion of its length (e.g., rectangular), combinations of those described herein, and any other material considered suitable for a particular embodiment. In the illustrated embodiment, each of the locking screw 12 and the tissue manipulator 14 is formed of polyetheretherketone. Alternative embodiments, however, can include a tissue manipulator that is formed of a material that is the same as, or different than, a material that forms a locking screw.

When assembled, the shaft 74 is partially disposed through the locking screw passageway 28 such that the shaft exterior thread 92 mates and interacts with the locking screw interior thread 36. The position of the tissue manipulator 14 relative to the locking screw 12 can be manipulated by moving the locking screw 12 relative to the tissue manipulator 14, the tissue manipulator 14 relative to the locking screw 12, and/or the locking screw 12 and the tissue manipulator 14 relative to one another. The mating configuration between the locking screw 12 and the tissue manipulator 14 is considered advantageous because it provides a mechanism for utilizing a single locking screw to secure the position of a tissue manipulator, which results in an overall reduction in the complexity and time required to implant a tissue fixation system. In addition, the mating configuration between the locking screw 12 and the tissue manipulator 14 is considered advantageous because it provides a mechanism for reducing, or eliminating, any element or feature, from extending beyond, or being positioned on, an outer surface of the wall to which the locking screw is intended to be attached (e.g., a surface of the thyroid cartilage).

While the locking screw 12 and tissue manipulator 14 have been illustrated as forming a tissue fixation system 10, a locking screw can be provided separately from a tissue manipulator individually, or combined with other components, systems, or elements. While the locking screw 12 and tissue manipulator 14 have been illustrated as forming a tissue fixation system 10, a tissue manipulator can be provided separately from a locking screw individually, or combined with other components, systems, or elements.

FIGS. 5, 6, 7, and 8 illustrate another example tissue fixation system 210. The tissue fixation system 210 is similar to the tissue fixation system 10 illustrated in FIGS. 1, 2, 3, and 4 and described above, except as detailed below. The tissue fixation system 210 includes a locking screw 212 and a tissue manipulator 214.

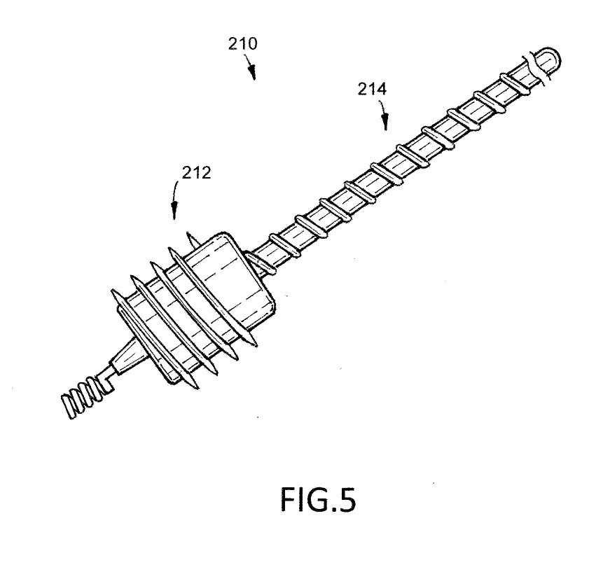

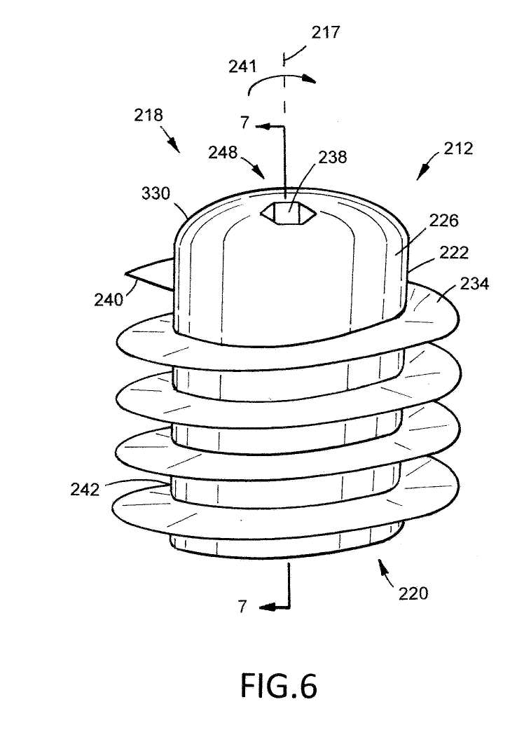

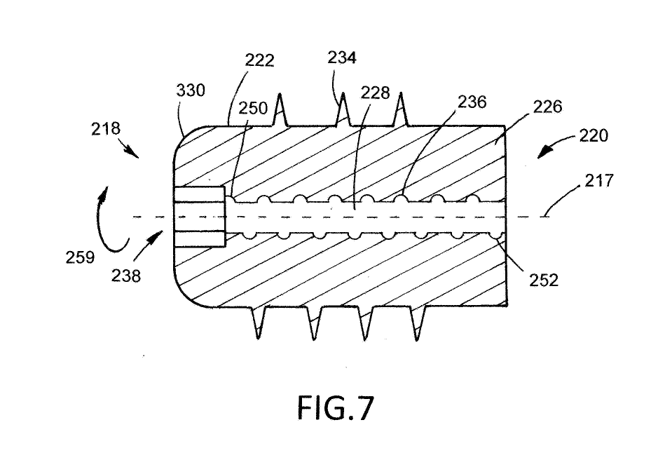

As shown in FIGS. 6 and 7, in the illustrated embodiment, the locking screw main body 226 defines an atraumatic locking screw first end 218, a locking screw exterior thread 234, a locking screw interior thread 236, and a recess 238. The locking screw exterior thread 234 extends from the locking screw exterior thread first end 240 to the locking screw exterior thread second end 242 and around the locking screw lengthwise axis 217 in a first direction 241. In the illustrated embodiment, the first direction 241 is clockwise. The locking screw interior thread 236 extends from the locking screw interior thread first end 250 to the locking screw interior thread second end 252 and around the locking screw lengthwise axis 217 in a second direction 259. In the illustrated embodiment, the second direction 259 is the same as the first direction 241 and is clockwise.

A curved surface 330 is defined between the locking screw first end 218 and the locking screw outer surface 222 and forms the atraumatic locking screw first end 218. This can be accomplished, for example, by machining the edge between the locking screw first end 218 and the locking screw outer surface 222. The recess 238 extends from the locking screw first end 218 toward the locking screw second end 220 and is in communication with the locking screw passageway 228. The recess 238 is a hexagonal prism and is sized and configured to receive a portion of a tissue manipulator, such as tissue manipulator 214, and/or a tool that is sized and configured to trim a portion of a tissue manipulator. In addition, the recess 238 defines an opening 248 that is sized and configured to receive a portion of a delivery tool, such as delivery tool 500 described in more detail herein, that is configured to engage the locking screw 212 and advance or withdraw the locking screw 212 into and out of another feature, element, or component, such as the thyroid cartilage of a patient.

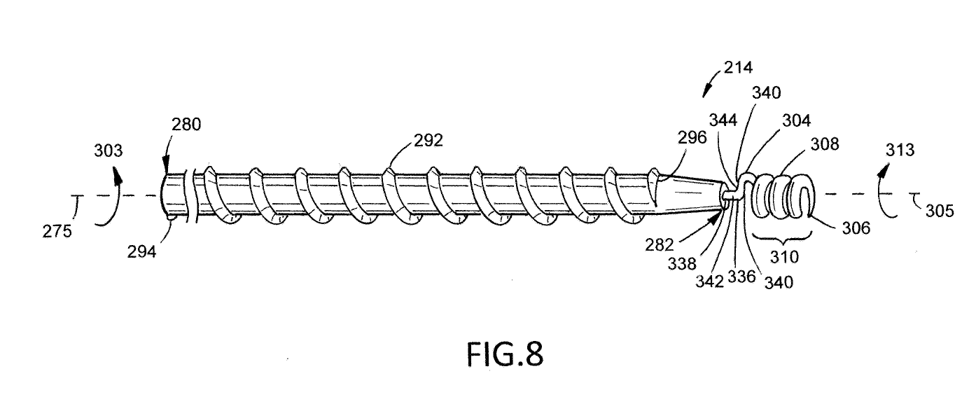

As shown in FIG. 8, in the illustrated embodiment, the shaft exterior thread 292 extends from the shaft exterior thread first end 294 to the shaft exterior thread second end 296 and around the shaft lengthwise axis 275 in a third direction 303 that is the same as the first direction 241. In the illustrated embodiment, the third direction 303 is clockwise. In the illustrated embodiment, the coil main body 308 defines an elongate member 336 and a plurality of coil turns 310. The elongate member 336 has an elongate member first end 338, an elongate member second end 340, an elongate member first portion 342, and an elongate member second portion 344. The elongate member first portion 342 extends from the shaft second end 282 to the elongate member second portion 344. The elongate member second portion 344 extends from the elongate member first portion 342 to the plurality of coil turns 310. The elongate member first portion 342 extends from the shaft second end 282 and away from the shaft first end 280. The elongate member second portion 344 extends from the elongate member first portion 342 at an angle of about 90 degrees relative to the elongate member first portion 342. The coil 276 extends from the elongate member second end 340, from the coil first end 304 to the coil second end 306, and around the coil lengthwise axis 305 in a fourth direction 313 that is the different than the first direction 241. In the illustrated embodiment, the fourth direction 313 is counterclockwise.

While the elongate member first portion 342 and elongate member second portion 344 have been illustrated as having a particular structural configuration, an elongate member first portion and elongate member second portion of a tissue manipulator can have any suitable structural configuration. Selection of a suitable structural configuration can be based on various considerations, including the structural configurations of the portion of the body to which a tissue manipulator is intended to be attached. For example, an elongate member first portion can be disposed at any suitable angle relative to a shaft and/or an elongate member second portion can be disposed at any suitable angle relative to an elongate member first portion. Example angles considered suitable between an elongate member first portion and a shaft and/or between an elongate member second portion and an elongate member first portion include angles equal to, less than, or greater than, 90 degrees, 45 degrees, 30 degrees, and any other angle considered suitable for a particular embodiment.

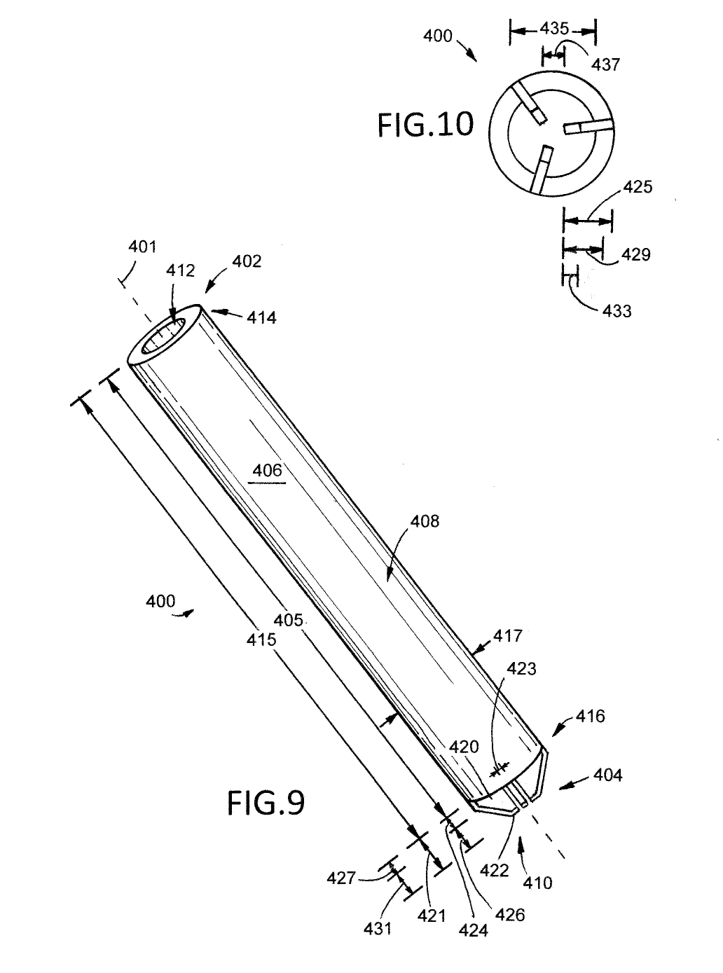

FIGS. 9 and 10 illustrate an example delivery tool 400 that can be used to implant a tissue fixation system, such as tissue fixation system 10, as described in more detail herein. The delivery tool 400 can be provided individually, or be included in a tissue fixation system, such as tissue fixation system 10.

In the illustrated embodiment, the delivery tool 400 has a delivery tool lengthwise axis 401, a delivery tool first end 402, a delivery tool second end 404, a delivery tool axial length 405, and a delivery tool main body 406 that defines an elongate member 408, a plurality of projections 410, and a delivery tool passageway 412 that extends from the delivery tool first end 402 to the delivery tool second end 404. The delivery tool axial length 405 extends from the delivery tool first end 402 to the delivery tool second end 404.

In the illustrated embodiment, the elongate member 408 has an elongate member first end 414, an elongate member second end 416, an elongate member length 415, and an elongate member outside diameter 417. The elongate member length 415 extends from the elongate member first end 414 to the elongate member second end 416. The elongate member outside diameter 417 is constant along the elongate member length 415. Each projection of the plurality of projections 410 extends from the elongate member second end 416 to the delivery tool second end 404 and has a projection first end 420, a projection second end 422, a projection length 421, a projection first portion 424, a projection second portion 426, and a projection thickness 423. The projection length 421 extends from the projection first end 420 to the projection second end 422.

The projection first portion 424 extends from the projection first end 420 toward the projection second end 422 and the projection second portion 426 extends from the projection first portion 424 to the projection second end 422. The projection first portion 424 has a projection first portion width 425 and a projection first portion length 427. The projection second portion 426 has a projection second portion width 429 and a projection second portion length 431. The projection second end 422 has a projection second end width 433. In the illustrated embodiment, the projection first portion width 425 is greater than the projection second portion width 429 and the projection second end width 433. The projection second portion width 425 decreases from the projection first portion 424 to the projection second end 422. The projection first portion length 427 is less than the projection second portion length 431. Each projection of the plurality of projections 410 is sized and configured to mate with a recess of a locking screw (e.g., recess of the plurality of recesses 38) such that delivery tool 400 can be used to advance and withdraw the locking screw into and out another feature, element, or component, such as the thyroid cartilage of a patient.

The delivery tool passageway 412 has a delivery tool passageway first inside diameter 435 and a delivery tool passageway second inside diameter 437. The delivery tool passageway first inside diameter 435 is disposed within the elongate member 408 and is greater than the shaft exterior thread major diameter of a tissue manipulator (e.g., shaft exterior thread major diameter 93). In the illustrated embodiment, the delivery tool passageway first inside diameter 435 extends from the elongate member first end 414 to the elongate member second end 416. The delivery tool passageway second inside diameter 437 is disposed between the plurality of projections 410 and is greater than the shaft exterior thread major diameter of a tissue manipulator (e.g., shaft exterior thread major diameter 93). In the illustrated embodiment, the delivery tool passageway second inside diameter 437 extends from the elongate member second end 416 to the delivery tool second end 404. The delivery tool passageway first inside diameter 435 is greater than the delivery tool passageway second inside diameter 437. Each of the delivery tool passageway first inside diameter 435 and delivery tool passageway second inside diameter 437 is sized and configured to receive a portion of a tissue manipulator (e.g., tissue manipulator 14).

While the delivery tool has been illustrated as having a particular structural arrangement, a delivery tool can have any suitable structural arrangement and selection of a suitable structural arrangement can be based on various considerations, such as the structural arrangement of a locking screw and/or a tissue manipulator that is intended be used with a delivery tool. Example structural arrangements considered suitable for a delivery tool include delivery tools that define a delivery tool passageway that has a constant inside diameter that extends from the delivery tool first end to the delivery tool second end, delivery tools that define a delivery tool passageway that has a constant inside diameter that extends from the elongate member first end to the elongate member second end, delivery tools that define a delivery tool passageway that has an inside diameter that varies from the elongate member first end to the elongate member second end or from the elongate member second end to the delivery tool second end, delivery tools that include a plurality of projections that each have a constant, or varied, width, length, or thickness, delivery tools that define a single projection that is sized and configured (e.g., hexagonal prism) to be received by a recess defined by a locking screw, and any other structural arrangement considered suitable for a particular embodiment.

A delivery tool 400 can be formed of any suitable material and selection of a suitable material to form a delivery tool according to a particular embodiment can be based on various considerations, including the material forming a locking screw and/or a tissue manipulator intended to be used with a delivery tool. Example materials considered suitable to form a delivery tool include rigid materials, semi-rigid materials, implant grade materials, implant grade rigid materials, implant grade semi-rigid materials, biocompatible materials, materials that can be made biocompatible, metals such as stainless steel, titanium, metal alloys, nickel-titanium alloy (e.g., Nitinol), thermoplastics, polymers, polyetheretherketone (PEEK), Pebax, nylon, polyethylene, high-density polyethylene (HDPE), high-performance polyethylene (HPPE), polyurethane, silicone, acrylonitrile butadiene styrene (ABS), polyoxymethylene (e.g., acetal), materials that have a round cross-sectional configuration, materials that have a rectangular, elongated, or square, cross-sectional configuration, materials that have a first cross-sectional configuration along a first portion of its length (e.g., round) and a second cross-sectional configuration along a second portion of its length (e.g., rectangular), combinations of those described herein, and any other material considered suitable for a particular embodiment. In the illustrated embodiment, the delivery tool 400 is formed of stainless steel. However, alternative embodiments can include a delivery tool that is formed of a material that is the same as, or different than, the material that forms a locking screw and/or a tissue manipulator. A delivery tool can be formed using any suitable technique or method of manufacture and selection of a suitable technique or method of manufacture to form a delivery tool can be based on various considerations, including the material that forms a locking screw and/or tissue manipulator intended to be used with a delivery tool. Example methods of manufacturing a delivery tool include those described herein, conventional machining techniques and methods of manufacture, 3D printing, injection molding, and any other technique or method of manufacture considered suitable for a particular embodiment.

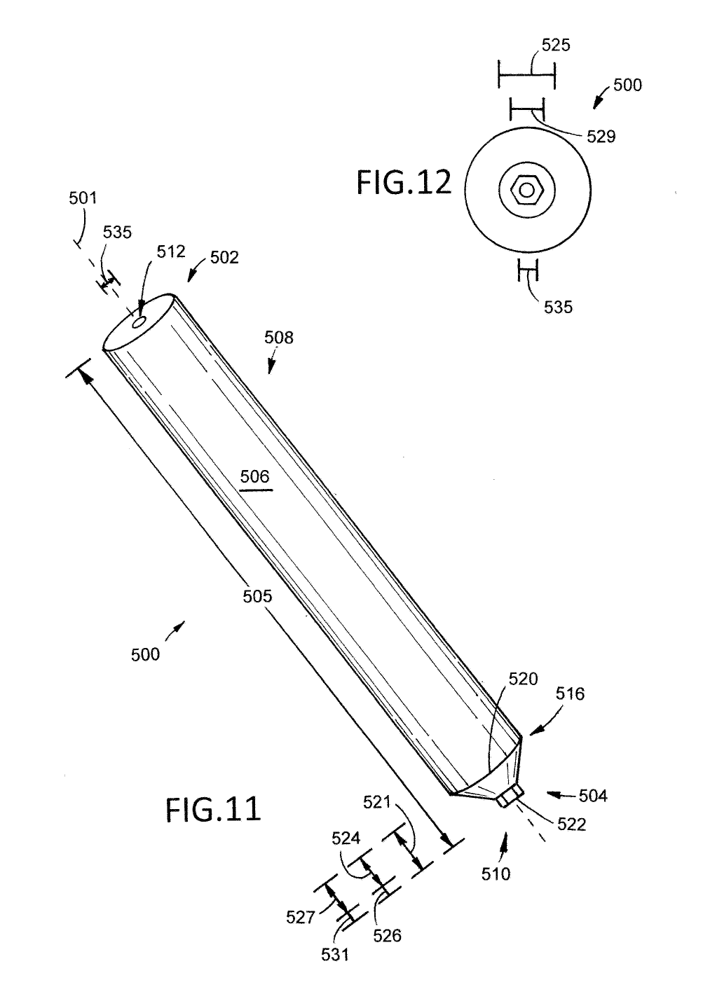

FIGS. 11 and 12 illustrate another example delivery tool 500 that can be used to implant a tissue fixation system, such as tissue fixation system 210, as described in more detail herein. The delivery tool 500 can be provided individually, or be included in a tissue fixation system, such as tissue fixation system 210.

In the illustrated embodiment, the delivery tool 500 has a delivery tool lengthwise axis 501, a delivery tool first end 502, a delivery tool second end 504, a delivery tool axial length 505, and a delivery tool main body 506 that defines an elongate member 508, a projection 510, and a delivery tool passageway 512 that extends from the delivery tool first end 502 to the delivery tool second end 504. The delivery tool axial length 505 extends from the delivery tool first end 502 to the delivery tool second end 504.

In the illustrated embodiment, the projection 510 extends from the elongate member second end 516 to the delivery tool second end 504 and has a projection first end 520, a projection second end 522, a projection length 521, a projection first portion 524, and a projection second portion 526. The projection length 521 extends from the projection first end 520 to the projection second end 522. The projection first portion 524 extends from the projection first end 520 toward the projection second end 522 and the projection second portion 526 extends from the projection first portion 524 to the projection second end 522. The projection first portion 524 has a projection first portion outside diameter 525 and a projection first portion length 527. The projection second portion 526 has a projection second portion outside diameter 529 and a projection second portion length 531. In the illustrated embodiment, the projection first portion outside diameter 525 is greater than the projection second portion outside diameter 529. The projection first portion outside diameter 525 decreases from the projection first end 520 to the projection second portion 526. The projection first portion length 527 is greater than the projection second portion length 531. In the illustrated embodiment, the projection second portion 526 is a hexagonal prism that is sized and configured to mate with a recess of a locking screw (e.g., recess 238) such that the delivery tool 500 can be used to advance and withdraw the locking screw into and out another feature, element, or component, such as the thyroid cartilage of a patient.

The delivery tool passageway 512 has a delivery tool passageway inside diameter 535 that is constant through the elongate member 508 and the projection 510. The delivery tool passageway inside diameter 535 is greater than the shaft exterior thread major diameter of a tissue manipulator (e.g., tissue manipulator 214). In the illustrated embodiment, the delivery tool passageway first inside diameter 535 extends from the delivery tool first end 502 to the delivery tool second end 504. The delivery tool passageway inside diameter 535 is sized and configured to receive a portion of a tissue manipulator (e.g., tissue manipulator 214).

Various methods of manufacturing a tissue manipulator are described herein. While the methods described herein are shown and described as a series of acts, it is to be understood and appreciated that the methods are not limited by the order of acts, as some acts may in accordance with these methods, occur in different orders, and/or concurrently with other acts described herein.

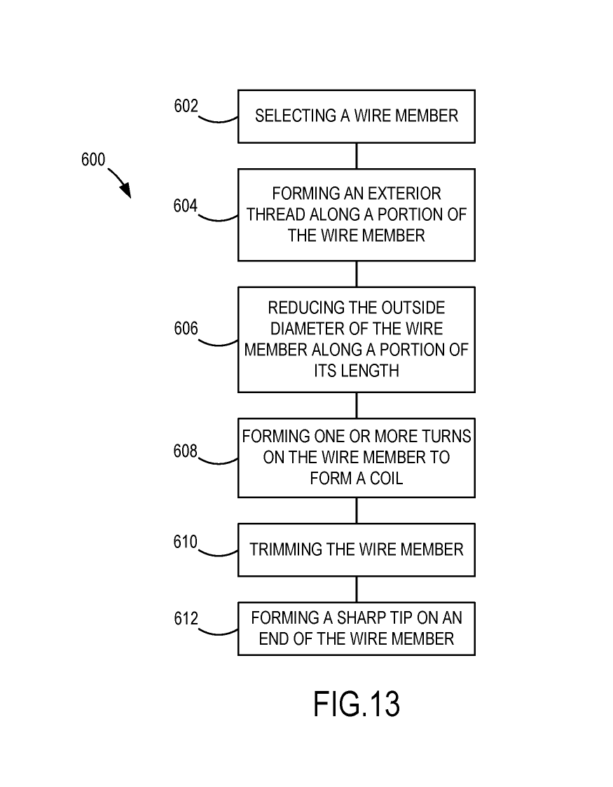

FIG. 13 is a schematic illustration of a method 600 of manufacturing a tissue manipulator.

An initial step 602 comprises selecting a wire member to form a tissue manipulator. The wire member has a wire member first end and a wire member second end. Another step 604 comprises forming an exterior thread along a portion of the axial length of the wire member. Another step 606 comprises reducing the outside diameter of the wire member along a portion of its axial length that extends from the wire member second end toward the wire member first end. Another step 608 comprises forming one or more turns along the portion of the axial length of the wire member that extends from the wire member second end toward the wire member first end to form a coil. Another step 610 comprises trimming the wire member to a desired length. Another step 612 comprises forming a sharpened tip on the wire member second end such that the wire member second end is sharp relative to the wire member first end.

Step 602 can be accomplished by selecting any suitable wire member and selection of a suitable wire member can be based on various considerations, such as the structural arrangement of a locking screw through which a tissue manipulator is intended to be passed. For example, round wire members that have a length at least, greater than, or equal to, about 12.7 centimeters long and an outside diameter between about 0.030 inches and about 0.060 inches are considered suitable. However, as described herein, use of other wire member sizes and configurations are considered suitable.

Step 604 can be accomplished using any suitable technique or method of forming an exterior thread along a portion of the axial length of the wire member and selection of a suitable technique or method of forming an exterior thread along a portion of the axial length of the wire member can be based on various considerations, such as the material(s) that forms the tissue manipulator. Examples of suitable techniques and methods of forming an exterior thread along a portion of the axial length of the wire member include thread rolling, grinding, cutting, chasing, and any other technique or method considered suitable for a particular embodiment. An exterior thread included on a tissue manipulator can be located along any suitable portion of the axial length and on any suitable portion of a tissue manipulator. For example, an exterior thread can be formed along a portion, or the entirety, of a shaft.

Step 606 can be accomplished using any suitable technique or method of reducing the outside diameter, or tapering the outside diameter, of a portion of the tissue manipulator length that extends from a location between the tissue manipulator first end and the tissue manipulator second end to the tissue manipulator second end. Selection of a suitable technique or method of reducing, or tapering, the outside diameter of a portion of the tissue manipulator length can be based on various considerations, such as the material(s) that forms the tissue manipulator. Examples of suitable techniques and methods for reducing, or tapering, the outside diameter of a portion of the tissue manipulator length include center-less grinding, electrolysis, acid-dipping, pulling the portion of the tissue manipulator length under tension, and any other suitable technique or method considered suitable for a particular embodiment.

Step 608 can be accomplished using any suitable technique or method of forming one or more turns along a portion of the length of a wire member to form a coil and selection of a suitable technique or method can be based on various considerations, such as the material(s) that forms the wire member. Examples of suitable techniques and methods of forming one or more turns along a portion of the length of a wire member to form a coil include using a mandrel, using a form, and any other suitable technique or method considered suitable for a particular embodiment. For example, the portion of the wire member intended to form a coil (e.g., the portion having a reduced or tapered outside diameter) can be pulled around a mandrel to produce a coil.

An optional step comprises adjusting the coil lengthwise axis to ensure that it is positioned relative to the shaft lengthwise axis as desired (e.g., coaxial, parallel to, at an angle to). Another optional step comprises adjusting the coil to ensure that it has the desired structural configuration (e.g., distance between turns, angle of turns). These optional steps can be accomplished by maintaining the position of the shaft and manipulating the position of the coil by applying an appropriate force on the coil (e.g., axial, torque), maintaining the position of the coil and manipulating the position of the shaft by applying an appropriate force on the shaft (e.g., axial, torque), or manipulating the position of the shaft while concurrently manipulating the position of the coil by applying an appropriate force on the shaft and coil (e.g., axial, torque).

Step 610 can be accomplished using any suitable technique or method of trimming a wire member and selection of a suitable technique or method can be based on various considerations, such as the material(s) that forms the wire member. Examples of suitable techniques and methods of trimming a wire member include cutting using a cutting tool (e.g., snips), grinding, and any other suitable technique or method considered suitable for a particular embodiment. For example, the shaft length and number of coil turns included on a tissue manipulator can be based on the anatomy of a patient (e.g., thickness of the arytenoid muscle, distance between thyroid cartilage and arytenoid muscle) and the number of coil turn on a tissue manipulator and/or the shaft length of a tissue manipulator can be trimmed based on the anatomy of the patient. Optional steps include trimming the shaft length, trimming the coil length, and/or trimming the number of coil turns.

Step 612 can be accomplished using any suitable technique or method of creating a sharpened tip on a wire member second end (e.g., tissue manipulator second end) and selection of a suitable technique or method can be based on various considerations, such as the material(s) that forms the wire member. Examples of suitable techniques and methods of creating a sharpened tip on a wire member include grinding, filing, electrolysis, and any other suitable technique or method considered suitable for a particular embodiment.

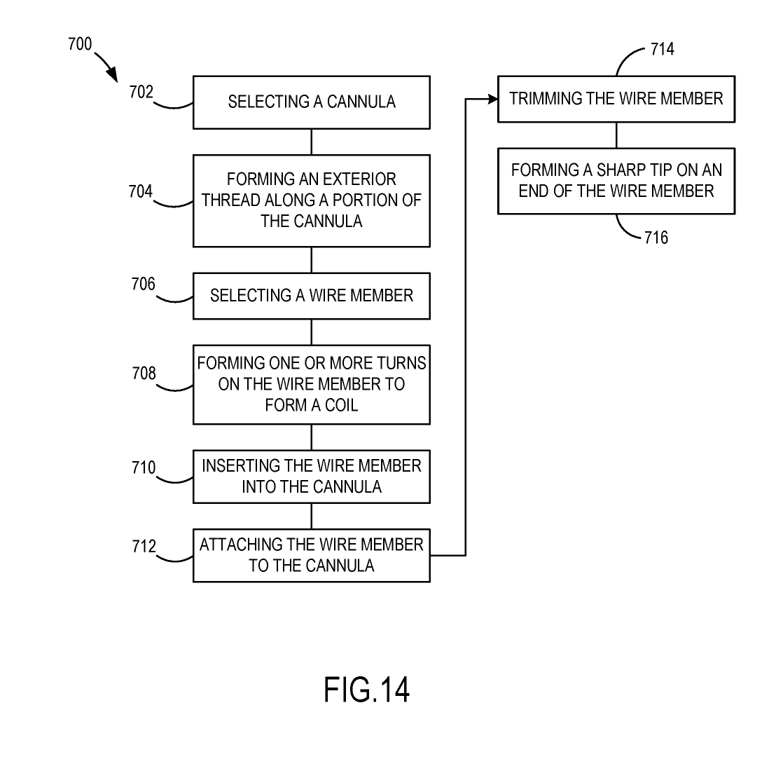

FIG. 14 is a schematic illustration of another method 700 of manufacturing a tissue manipulator.

A step 702 comprises selecting a cannula. Another step 704 comprises forming an exterior thread along a portion of the axial length of the cannula. Another step 706 comprises selecting a wire member. The wire member has a wire member first end and a wire member second end. Another step 708 comprises forming one or more turns along a portion of the axial length of the wire member that extends from the wire member second end toward the wire member first end to form a coil. Another step 710 comprises inserting the wire member into a lumen defined by the cannula. Another step 712 comprises attaching the wire member to the cannula. Another step 714 comprises trimming the wire member to a desired length. Another step 716 comprises forming a sharpened tip on the wire member second end such that the wire member second end is sharp relative to the wire member first end.

Step 702 can be accomplished by selecting any suitable cannula and selection of a suitable cannula can be based on various considerations, such as the structural arrangement of a locking screw through which a tissue manipulator is intended to be passed. Alternatively, step 702 can comprise selecting an elongate rod. This alternative step can be accomplished by selecting any suitable elongate rod and selection of a suitable elongated rod can be based on various considerations, such as the structural arrangement of a locking screw through which a tissue manipulator is intended to be passed.

Step 704 can be accomplished using any suitable technique or method of forming an exterior thread along a portion of the axial length of the cannula and selection of a suitable technique or method of forming an exterior thread along a portion of the axial length of the cannula can be based on various considerations, such as the material(s) that forms the cannula. Examples of suitable techniques and methods of forming an exterior thread along a portion of the axial length of the cannula include thread rolling, grinding, cutting, chasing, and any other technique or method considered suitable for a particular embodiment. In embodiments in which step 702 comprises selecting an elongate rod, step 704 can alternatively comprise forming an exterior thread along a portion of the axial length of the elongate rod. This alternative step can be accomplished using any suitable technique or method of forming an exterior thread along a portion of the axial length of the elongate rod and selection of a suitable technique or method of forming an exterior thread along a portion of the axial length of the elongate rod can be based on various considerations, such as the material(s) that forms the elongate rod. Examples of suitable techniques and methods of forming an exterior thread along a portion of the axial length of the elongate rod include thread rolling, grinding, cutting, chasing, and any other technique or method considered suitable for a particular embodiment.

Step 706 can be accomplished by selecting any suitable wire member and selection of a suitable wire member can be based on various considerations, such as the structural arrangement of a locking screw through which a tissue manipulator is intended to be passed and/or the structural arrangement of the cannula and/or elongate rod to which the wire member is intended to be attached.

Step 708 can be accomplished as described herein with respect to step 608.

Step 710 can be accomplished by inserting the end of the wire member opposite of the end that forms the one or more turns into the lumen defined by the cannula such that the end is disposed within the lumen. Alternatively, in embodiments in which step 702 comprises selecting an elongate rod, step 710 comprises positioning the end of the wire member opposite of the end that forms the one or more turns adjacent to an end of the elongate rod (e.g., such that the ends contact one another).

Step 712 can be accomplished using any suitable technique or method of attachment and selection of a suitable technique or method of attachment to use between a cannula and a wire member can be based on various considerations, such as the material(s) that forms the cannula and/or the wire member. Example techniques and methods of attachment considered suitable between a cannula and a wire member include welding, fusing, using adhesive, and any other technique or method considered suitable for a particular embodiment. Alternatively, in embodiments in which step 702 comprises selecting an elongate rod, step 712 comprises attaching the wire member to the elongate rod. This alternative step can be accomplished using any suitable technique or method of attachment and selection of a suitable technique or method of attachment to use between an elongate rod and a wire member can be based on various considerations, such as the material(s) that forms the elongate rod and/or the wire member. Example techniques and methods of attachment considered suitable between an elongate rod and a wire member include welding, fusing, using adhesive, and any other technique or method considered suitable for a particular embodiment.

An optional step comprises adjusting the coil lengthwise axis to ensure that it is positioned relative to the shaft lengthwise axis as desired (e.g., coaxial, parallel to, at an angle to). Another optional step comprises adjusting the coil to ensure that it has the desired structural configuration (e.g., distance between turns, angle of turns). These optional steps can be accomplished by maintaining the position of the shaft and manipulating the position of the coil by applying an appropriate force on the coil (e.g., axial, torque), maintaining the position of the coil and manipulating the position of the shaft by applying an appropriate force on the shaft (e.g., axial, torque), or manipulating the position of the shaft while concurrently manipulating the position of the coil by applying an appropriate force on the shaft and coil (e.g., axial, torque).

Step 714 can be accomplished as described herein with respect to step 610.

Step 716 can be accomplished as described herein with respect to step 612.

While particular methods of forming a tissue manipulator have been described herein, a locking screw and tissue manipulator can be formed using any suitable technique or method of manufacture and selection of a suitable technique or method of manufacture can be based on various considerations, including the material that forms a locking screw and/or tissue manipulator. Example techniques and methods of manufacturing considered suitable to form a locking screw and/or tissue manipulator include those described herein, conventional machining techniques and methods of manufacture, 3D printing, injection molding, and any other technique or method of manufacture considered suitable for a particular embodiment.

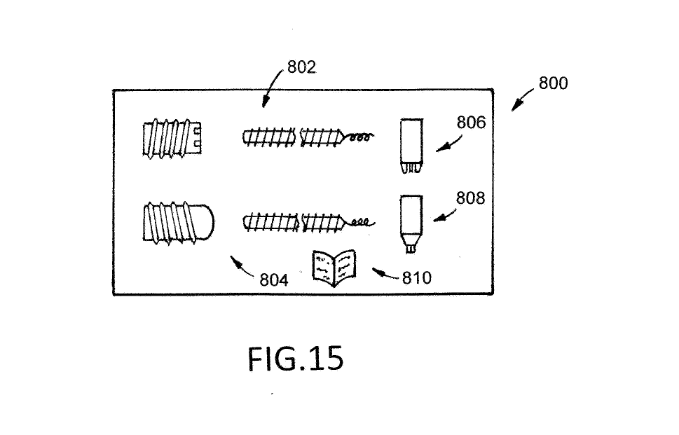

FIG. 15 illustrates an exemplary kit 800 comprising a first tissue fixation system 802 according to an embodiment, such as tissue fixation system 10 illustrated in FIGS. 1, 2, 3, and 4; a second tissue fixation system 804 according to an embodiment, such as tissue fixation system 210 illustrated in FIGS. 5, 6, 7, and 8; a first delivery tool 806 according to an embodiment, such as delivery tool 400 illustrated in FIGS. 9 and 10; a second delivery tool 808 according to an embodiment, such as delivery tool 500 illustrated in FIGS. 11 and 12; and instructions for use 810.

While kit 800 has been illustrated as including two tissue fixation systems 802, 804, and two delivery tools 806, 808, any suitable number, and type, of tissue fixation systems and/or delivery tools can be included in a kit. Selection of a suitable number of tissue fixation systems and/or delivery tools to include in a kit according to a particular embodiment can be based on various considerations, such as the treatment intended to be performed. Examples of suitable numbers of tissue fixation systems and/or delivery tools to include in a kit include at least one, one, two, a plurality, three, four and any other number considered suitable for a particular embodiment.

Furthermore, while tissue fixation system 10, tissue fixation system 210, delivery tool 400, and delivery tool 500 have been illustrated as included in kit 802, any suitable tissue fixation system and/or delivery tool can be included in a kit. Selection of a suitable tissue fixation system and/or delivery tool to include in a kit according to a particular embodiment can be based on various considerations, such as the treatment intended to be performed. Examples of tissue fixation systems considered suitable to include in a kit include tissue fixation system 10, tissue fixation system 210, variations of the tissue fixation systems described herein, and/or any other tissue fixation system considered suitable for a particular embodiment. Examples of delivery tools considered suitable to include in a kit include delivery tool 400, delivery tool 500, variations of the delivery tools described herein, and/or any other delivery tool considered suitable for a particular embodiment.

In an alternative embodiment, a kit can include a first tissue fixation system, a second tissue fixation system, a first delivery tool, and a second delivery tool. The second tissue fixation system can be scaled relative to the first tissue fixation system. A kit that includes one or more tissue fixation systems that are scaled relative to one another provides a mechanism for selecting a tissue fixation system that best fits a particular patient's anatomy. For example, the first tissue fixation system can be sized and configured for a pediatric patient, the second tissue fixation system can be 2:1 scale of the first tissue fixation system and be sized and configured for teenage patient, or the second tissue fixation system can be 4:1 scale of the first tissue fixation system and be sized and configured for an adult patient. The first delivery tool can be sized and configured to interact with the first tissue fixation system. The second delivery tool can be sized and configured to interact with the second tissue fixation system. In embodiments in which more than one tissue fixation system is included in a kit, a second tissue fixation system can be scaled relative to a first tissue fixation system based on any suitable proportional ratio. Selection of a suitable amount to scale a second tissue fixation system relative to a first tissue fixation system can be based on various considerations, including the treatment intended to be performed. For example, a second tissue fixation system can be scaled such that it is 1:1, 2:1, 3:1, 4:1, 5:1, 6:1, 7:1 relative to a first tissue fixation system, between about 1:1 and about 7:1 relative to a first tissue fixation system, and any other proportional ratio considered suitable for a particular embodiment.

Various methods of treatment are described herein. While the methods described herein are shown and described as a series of acts, it is to be understood and appreciated that the methods are not limited by the order of acts, as some acts may in accordance with these methods, occur in different orders, and/or concurrently with other acts described herein.

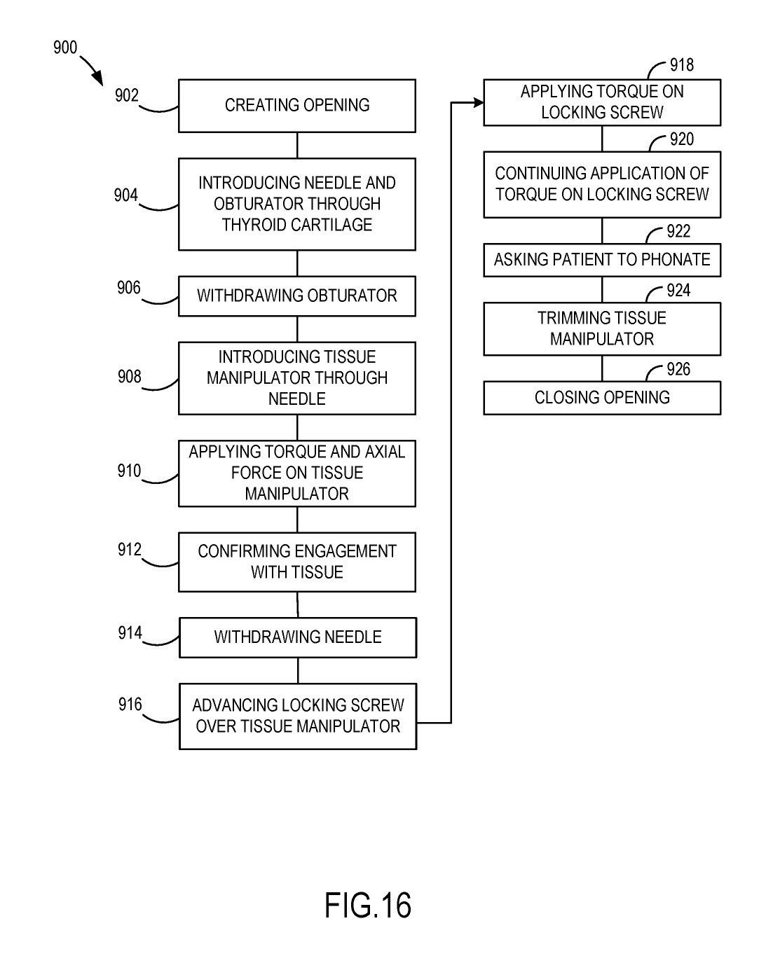

FIG. 16 is a schematic illustration of an example method of treatment 900 using a tissue fixation system and a delivery tool. In the example described herein, the method of treatment comprises a method of repositioning an arytenoid muscle resulting in repositioning of a vocal cord.

A step 902 comprises creating an opening in the body over the vocal cord intended to be treated to access the thyroid cartilage. Another step 904 comprises introducing a needle and an obturator through the opening and the thyroid cartilage and into the arytenoid muscle. Another step 906 comprises withdrawing the obturator while maintaining the position of the needle. Another step 908 comprises passing a tissue manipulator through the needle until it contacts the arytenoid muscle. Another step 910 comprises applying torque on the tissue manipulator while applying an axial force toward the arytenoid muscle such that the tissue manipulator rotates and engages the arytenoid muscle. Another step 912 comprises confirming engagement of the tissue manipulator with the arytenoid muscle. Another step 914 comprises withdrawing the needle from the tissue and thyroid cartilage while maintaining position of the tissue manipulator. Another step 916 comprises advancing a locking screw that has a locking screw first end, a locking screw second end, and a locking screw main body that defines a locking screw passageway over the tissue manipulator such that a portion of the tissue manipulator is disposed within the locking screw passageway. Another step 918 comprises applying torque on the locking screw while maintaining the position of the tissue manipulator such that the locking screw rotates relative to the tissue manipulator and engages the thyroid cartilage. Another step 920 comprises continuing the application of torque on the locking screw while maintaining the position of the tissue manipulator until the locking screw first end is substantially flush with a surface of the thyroid cartilage. Another step 922 comprises asking the patient to phonate to evaluate the position of the vocal cord and to determine whether adjustments are needed. Another step 924 comprises trimming the tissue manipulator. Another step 926 comprises closing the opening made in the body.

Step 902 can be accomplished using any suitable medical device (e.g., scalpel) and by creating the opening on the side of the vocal cord that is being treated and over the thyroid cartilage. Step 902 can be accomplished by creating an opening that is sized and configured to allow a portion of a tissue fixation system and/or delivery tool (e.g., tissue fixation system 10, tissue fixation system 210, delivery tool 400, delivery tool 500) to pass through the opening.

An optional step comprises administering medication near a point of treatment. This optional step can be accomplished prior to step 902 by administering any suitable medication at any suitable location on the body of a patient. For example, medication can be administered on the side of the vocal cord being treated and over the thyroid cartilage. Any suitable medication such as a local anesthetic, combination of local anesthetic and vasoconstrictor (e.g., 1% lidocaine, with 1:100,000 epinephrine), and/or general anesthetic can be used and administered in any suitable manner, such as subcutaneously using a convention syringe.

Another optional step that can be completed prior to step 902 comprises asking the patient to phonate. This optional step can be accomplished by providing instructions to a patient to phonate. Another optional step that can be completed prior to step 902, or subsequent to step 902, comprises determining how a vocal cord needs to be modified for effective treatment based on patient's anatomy and/or phonation. This step can be accomplished by visualizing the vocal cord using a scope that is partially disposed in a patient's larynx and/or by listening to the patient phonate.

Another optional step comprises dissecting the underlying soft tissues to expose the thyroid cartilage. This optional step can be accomplished subsequent to step 902 using any suitable medical device such that the thyroid cartilage is exposed. For example, this optional step can be accomplished using one or more of a needle, probe, pick, scalpel, forceps, tweezers, scissors, or any other device considered suitable for a particular procedure.

Another optional step that can be completed subsequent to step 902 comprises creating one or more pilot holes through the thyroid cartilage sized and configured to receive a portion of a tissue fixation device and/or delivery tool (e.g., tissue fixation system 10, tissue fixation system 210, delivery tool 400, delivery tool 500). This optional step can be accomplished using any suitable drill and a drill bit that has a first outside diameter. A drill bit used to complete any step of the methods described herein can have any suitable outside diameter, and skilled artisans will be able to select a suitable outside diameter for a drill bit according to a particular embodiment based on various considerations, including the structural arrangement at a point of treatment. Example outside diameters considered suitable for a drill bit include outside diameters that are equal to, less than, or greater than the locking screw exterior thread minor diameter of a locking screw (e.g., locking screw exterior thread minor diameter 33). When a pilot hole is created, it is considered advantageous to use a drill bit with an outside diameter that is less than, or equal to, the locking screw exterior thread minor diameter such that a friction fit can be accomplished between the locking screw and the thyroid cartilage.

Step 904 can be accomplished using any needle and obturator considered suitable for a particular embodiment and selection of a suitable needle and obturator can be based on various considerations, such as the structural configuration of a tissue fixation system intended to be implanted. The needle comprises a needle first end and a sharp needle second end and defines a lumen that extends from the needle first end to the needle second end. The obturator comprises an obturator first end and a sharp obturator second end and may optionally contain a light source. Alternatively, the obturator second end can be rounded or blunt. The obturator is disposed within the needle lumen. Alternatively, step 904 can comprise introducing a needle through the opening and the thyroid cartilage and into the arytenoid muscle and omit the inclusion of an obturator. Alternatively, step 904 can comprise advancing the needle and obturator, or an obturator, through the opening and a pre-drilled pilot hole and into the arytenoid muscle. Any suitably sized needle can be used to complete the methods described herein. Example needles considered suitable include those that define a lumen that is sized and configured to receive a portion of a tissue manipulator, that define a lumen that has an inside diameter equal to, greater than, less than, or about 0.042 inches, 0.05 inches, needles that have an outside diameter equal to, less than, or about the major diameter of a locking screw, or the minor diameter of a locking screw, 16 gage needles, needles between 13 gage and 25 gage, and any other needle considered suitable for a particular embodiment.

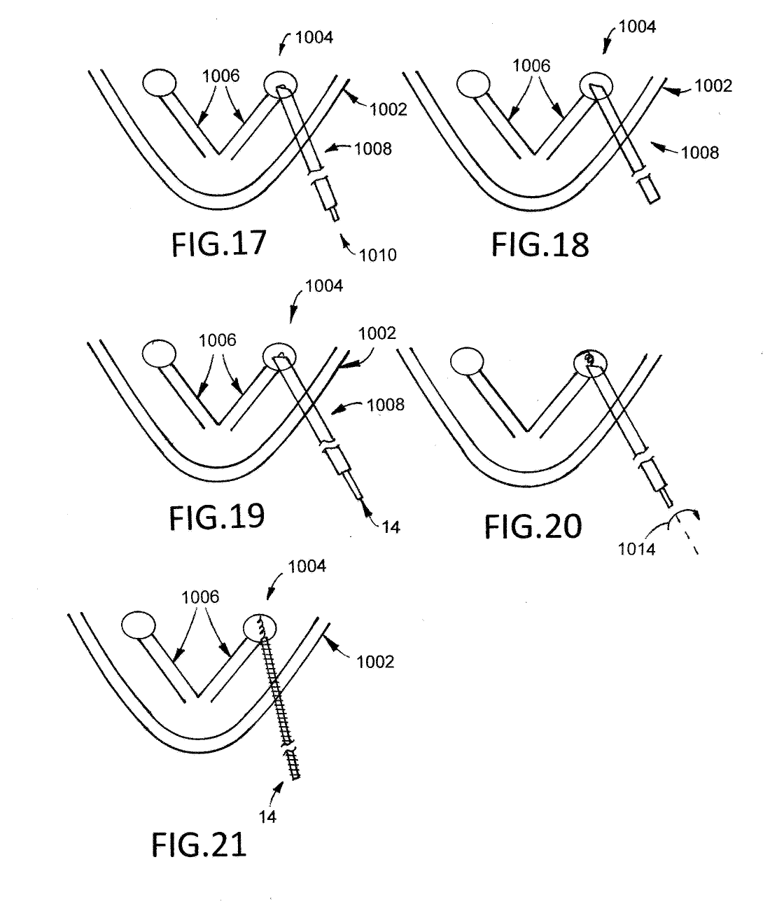

Step 904 can be accomplished by locating the thyroid cartilage, positioning the needle second end and the obturator second end on the thyroid cartilage, and applying an axial force toward the thyroid cartilage on the needle and obturator such that it passes through the thyroid cartilage and into the arytenoid muscle (e.g., translaryngeal insertion of needle and obturator). It is considered advantageous to introduce the needle and obturator into the arytenoid muscle from the anterior to the posterior and to stop the application of the axial force on the needle and obturator before the needle second end and/or obturator second end penetrates the posterior surface of the arytenoid muscle. FIG. 17 illustrates a superior view of the thyroid cartilage 1002, the arytenoid muscle 1004, and vocal cords 1006 with the needle 1008 and obturator 1010 disposed through the thyroid cartilage 1002 and partially disposed within the arytenoid muscle 1004. Optionally, step 904 can be accomplished using a laryngoscope disposed within a portion of the larynx to facilitate direct visualization of the needle and/or obturator. An optional step comprises confirming the placement of the needle second end and/or obturator second end using the laryngoscope and/or transillumination.

Step 906 can be accomplished by maintaining the position of the needle and applying an axial force on the obturator away from the thyroid cartilage until the obturator becomes free of the needle lumen. FIG. 18 illustrates the needle 1008 disposed through the thyroid cartilage 1002 and partially disposed within the arytenoid muscle 1004 and the obturator 1010 withdrawn from the needle lumen. In embodiments in which an obturator is not disposed within the needle lumen, step 906 can be omitted from method 900.

Step 908 can be accomplished by introducing the tissue manipulator second end into the needle lumen and applying an axial force on the tissue manipulator toward the thyroid cartilage until the tissue manipulator second end contacts the arytenoid muscle (e.g., inside the rearmost end of the arytenoid muscle). Step 908 can be accomplished by applying an axial force on any suitable portion (e.g., shaft) of any suitable tissue manipulator according to an embodiment, such as the embodiments described herein. Selection of a suitable tissue manipulator to pass through a needle lumen can be based on various considerations, including the structural arrangement of the arytenoid muscle being treated. Example tissue manipulators considered suitable include tissue manipulator 14, tissue manipulator 214, and any other tissue manipulator considered suitable for a particular embodiment. FIG. 19 illustrates a superior view of the thyroid cartilage 1002 and the arytenoid muscle 1004 with the needle 1008 and tissue manipulator 14 disposed through the thyroid cartilage 1002 and partially disposed within the arytenoid muscle 1004. In the method of treatment 900, the tissue manipulator 14 illustrated and described with respect to FIGS. 1, 2, 3, and 4 has been illustrated as being releasably attached to the arytenoid muscle. However, alternative embodiments can comprise use of tissue manipulator 214 illustrated and described with respect to FIGS. 5, 6, 7, and 8.

Step 910 can be accomplished by applying torque to any suitable portion (e.g., shaft) of the tissue manipulator while applying an axial force toward the arytenoid muscle such that the tissue manipulator rotates and engages the arytenoid muscle (e.g., sufficient purchase in the arytenoid is achieved). Depending on the direction in which the turns of the coil of the tissue manipulator are configured, torque can be applied to the tissue manipulator in either a clockwise or counterclockwise direction about the shaft lengthwise axis of the tissue manipulator. Arrow 1014 illustrated in FIG. 20 shows the application of torque on the tissue manipulator 14 in a clockwise direction. This step is completed until approximately 3 or 4 turns of the tissue manipulator 14 are engaged with the tissue and such that the tissue manipulator 14 does not extend through the posterior surface of the arytenoid muscle. In embodiments in which tissue manipulator 214 is used to complete the method of treatment, a counterclockwise torque will be applied on the tissue manipulator 214 until approximately 3 or 4 turns of the tissue manipulator 214 are engaged with the tissue and such that the tissue manipulator 214 does not extend through the posterior surface of the arytenoid muscle.

Step 912 can be accomplished by visualizing the vocal cord using a scope that is partially disposed in a patient's larynx, listening to the patient phonate, and/or by tactile feedback from the tissue manipulator as the tissue manipulator is moved axially relative to the thyroid cartilage.

Step 914 can be accomplished by maintaining the position of the tissue manipulator and applying an axial force away from the thyroid cartilage on the needle until the needle is free of the thyroid cartilage. FIG. 21 illustrates the tissue manipulator 12 disposed through the thyroid cartilage 1002 and partially disposed within the arytenoid muscle 1004 and the needle withdrawn from the thyroid cartilage 1002. An optional step that can be completed subsequent to step 914 comprises positioning the tissue manipulator at a desired location within the opening created in the thyroid cartilage and creating a marking indicia on the tissue manipulator relative to the thyroid cartilage such that a proper placement of the locking screw can be accomplished by positioning the marking indicia substantially flush, or flush, a surface of the thyroid cartilage.

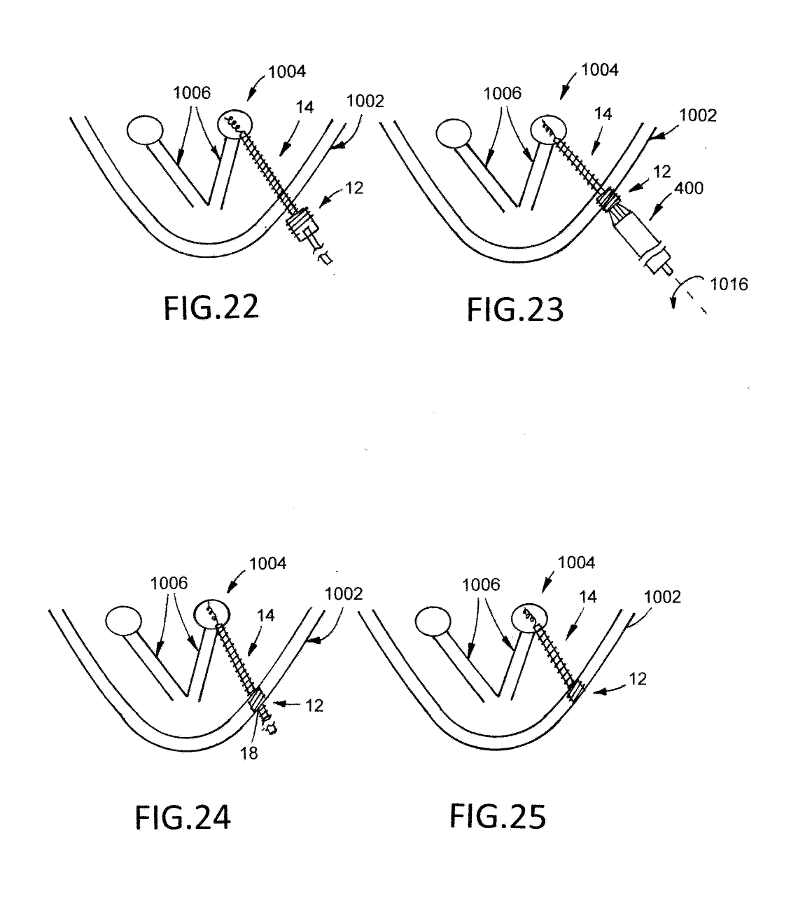

Step 916 can be accomplished by applying an axial force on the locking screw toward the tissue manipulator such that the tissue manipulator first end is passed through the locking screw first opening and is passed through the locking screw passageway. FIG. 22 illustrates a superior view of the thyroid cartilage 1002 and the arytenoid muscle 1004 with the tissue manipulator 14 disposed through the locking screw 12. In the method of treatment 900, the locking screw 12 illustrated and described with respect to FIGS. 1, 2, 3, and 4 has been illustrated as being used to complete the method of treatment 900. However, alternative embodiments can comprise use of locking screw 212 illustrated and described with respect to FIGS. 5, 6, 7, and 8.

Step 918 can be accomplished using any suitable delivery tool, device, or component and by applying torque to any suitable portion (e.g., locking screw first end) of the locking screw while maintaining the position of the tissue manipulator such that the locking screw exterior thread engages the thyroid cartilage and the locking screw interior thread mates and interacts with the shaft exterior thread of the tissue manipulator. Depending on the direction in which the locking screw exterior thread is disposed on the locking screw, torque can be applied to the locking screw in either a clockwise or counterclockwise direction about the locking screw lengthwise axis. Arrow 1016 illustrated in FIG. 23 shows the application of torque on the locking screw 12 in a counterclockwise direction using delivery tool 400. In embodiments in which locking screw 212 is used to complete the method of treatment, a clockwise torque will be applied on the locking screw 212 and can be accomplished using a delivery tool, such as delivery tool 500.

Step 920 can be accomplished by applying torque to any suitable portion (e.g., locking screw first end) of the locking screw while maintaining the position of the tissue manipulator until the locking screw first end is disposed substantially flush with a surface of the thyroid cartilage. Depending on the direction in which the locking screw exterior thread is disposed on the locking screw, torque can be applied to the locking screw in either a clockwise or counterclockwise direction about the locking screw lengthwise axis. FIG. 24 illustrates the locking screw first end 18 substantially flush with a surface of the thyroid cartilage 1002. While step 920 has been described as being accomplished such that the locking screw first end is substantially flush with a surface of the thyroid cartilage, a locking screw first end can be positioned at any suitable location relative to a surface of the thyroid cartilage and selection of a suitable location can be based on various considerations, including the treatment being performed and the desired phonation intended to be achieved. Example locations considered suitable to position a locking screw first end include substantially flush with a surface of the thyroid cartilage, within the wall of the thyroid cartilage, outside of the wall of the thyroid cartilage, and any other location considered suitable for a particular embodiment. As used herein, the phrase “substantially flush” refers to the locking screw first end being within one quarter, less than one quarter, one eighth, or less than one eighth, of a locking screw axial length relative to a surface of the thyroid cartilage, or a locking screw first end being within 1 millimeter, 2 millimeters, 3 millimeters, between about 0.5 millimeters and about 3.5 millimeters of a surface of the thyroid cartilage.

It is considered advantageous to include a shaft exterior thread that is disposed on a locking screw in a direction that it opposite the direction the coil extends at least because such a configuration provides a mechanism for maintaining the position of the tissue manipulator (e.g., coil) during use. For example, such a configuration provides friction and physical interaction between the tissue manipulator and the locking screw that prevents displacement of the coil after implantation.

Step 922 can be accomplished by providing instructions to a patient to phonate.

An optional step comprises adjusting the position of the tissue manipulator. This optional step can be accomplished by applying a torque on any suitable portion of the shaft of the tissue manipulator, the locking screw, or both the tissue manipulator and locking screw, to position the arytenoid muscle in a desired location that can be based on the patient's anatomy or that results in a desired phonation. For example, if the coil pitch is different than the shaft exterior thread pitch then the threaded differential provides a mechanism for retracting or advancing the tissue by rotating the tissue manipulator shaft. Alternatively, if the coil pitch and the shaft exterior thread pitch are the same, but they extend in opposition directions, then rotation of the tissue manipulator shaft provides a mechanism for axial displacement of the tissue.

Step 924 can be accomplished using any suitable technique or method of trimming a tissue manipulator and selection of a suitable technique or method can be based on various considerations, such as the material(s) that forms the tissue manipulator. Examples of suitable techniques and methods of trimming a tissue manipulator include cutting, using cutting tools (e.g., snips), grinding, and any other suitable technique or method considered suitable for a particular embodiment. FIG. 25 illustrates a trimmed tissue manipulator 14 disposed through the locking screw 12. For example, it is considered advantageous to trim the tissue manipulator such that the newly created tissue manipulator first end is disposed substantially flush with the locking screw first end, is disposed with a recess formed by the locking screw, or such that a portion of the tissue manipulator that extends from the newly created tissue manipulator first end toward the tissue manipulator second end can be bent relative to the remaining portion of the tissue manipulator and disposed within a recess formed by the locking screw. Alternatively, a tissue manipulator can be trimmed such that a portion of the tissue manipulator is disposed outside of a recess defined by a locking screw such that adjustments to the tissue manipulator can be made.

An optional step that can be completed subsequent to the completion of step 924 comprises asking the patient to phonate to evaluate the position of the vocal cord and to determine whether adjustments are needed. This optional step can be accomplished by providing instructions to a patient to phonate.

Step 926 can be accomplished using any suitable device and/or method, such as by suturing the opening created in step 902.

While method 900 has been described with respect to manipulating the position of an arytenoid muscle, any of the steps, alternative steps, and/or optional steps described herein can be utilized to treat any suitable tissue and a tissue fixation system can be attached to any suitable wall or feature of a patient. Selection of a suitable point of treatment to implant a tissue fixation system according to an embodiment can be based on various considerations, including the treatment intended to be performed and the structural arrangement at the treatment site.

Those with ordinary skill in the art will appreciate that various modifications and alternatives for the described and illustrated embodiments can be developed in light of the overall teachings of the disclosure. Accordingly, the particular arrangements disclosed are intended to be illustrative only and not limiting as to the scope of the invention, which is to be given the full breadth of the appended claims and any and all equivalents thereof.

What is claimed is:

1. A tissue fixation system comprising:
a locking screw having a locking screw lengthwise axis, a locking screw first end, a locking screw second end, a locking screw axial length extending from the locking screw first end to the locking screw second end, and a locking screw main body defining a locking screw passageway extending from the locking screw first end to the locking screw second end, a locking screw exterior thread, and a locking screw interior thread, the locking screw exterior thread extending along a portion of the locking screw axial length and having a locking screw exterior thread first end and a locking screw exterior thread second end, the locking screw interior thread disposed within the locking screw passageway and extending along a portion of the locking screw axial length and having a locking screw interior thread first end and a locking screw interior thread second end, the locking screw exterior thread extending from the locking screw exterior thread first end to the locking screw exterior thread second end and around the locking screw lengthwise axis in a first direction, the locking screw interior thread extending from the locking screw interior thread first end to the locking screw interior thread second end and around the locking screw lengthwise axis in a second direction that is the same as the first direction; and
a tissue manipulator having a tissue manipulator first end, a tissue manipulator second end, a tissue manipulator axial length extending from the tissue manipulator first end to the tissue manipulator second end, a shaft, and a coil, the shaft partially disposed through the locking screw passageway and having a shaft lengthwise axis, a shaft first end, a shaft second end, a shaft exterior surface, and a shaft main body extending from the shaft first end to the shaft second end and defining a shaft exterior thread extending along a portion of the tissue manipulator axial length, the shaft exterior thread sized and configured to mate with the locking screw interior thread and having a shaft exterior thread first end and a shaft exterior thread second end, the shaft exterior thread extending from the shaft exterior thread first end to the shaft exterior thread second end and around the tissue manipulator lengthwise axis in a third direction that is the same as the first direction, the coil extending from the shaft second end to the tissue manipulator second end and around the tissue manipulator lengthwise axis in a fourth direction that is different than the first direction.

2. The tissue fixation system of claim 1, wherein the first direction is clockwise.

3. The tissue fixation system of claim 1, wherein the first direction is counterclockwise.

4. The tissue fixation system of claim 1, wherein the coil defines a plurality of coil turns.

5. The tissue fixation system of claim 1, wherein the locking screw exterior thread first end is disposed between the locking screw first end and the locking screw second end;
wherein the locking screw exterior thread second end is disposed between the locking screw exterior thread first end and the locking screw second end.

6. The tissue fixation system of claim 1, wherein the shaft has a shaft outside diameter; and
wherein the coil has a coil outside diameter that is less than the shaft outside diameter.

7. The tissue fixation system of claim 1, wherein the shaft has a shaft first portion, and a shaft second portion, the shaft first portion extending from the shaft first end toward the shaft second end and having a shaft first portion outside diameter, the shaft second portion extending from the shaft first portion to the shaft second end and having a shaft second portion outside diameter that is less than the shaft first portion outside diameter.

8. The tissue fixation system of claim 7, wherein the shaft first portion outside diameter tapers to the shaft second portion outside diameter along a portion of the tissue manipulator axial length.

9. The tissue fixation system of claim 1, wherein the locking screw main body defines a recess extending from the locking screw first end toward the locking screw second end and in communication with the locking screw passageway.

10. The tissue fixation system of claim 1, wherein the shaft exterior thread has a shaft exterior thread pitch; and
wherein the coil has a coil pitch that is the same as the thread pitch.

11. The tissue fixation system of claim 1, wherein the locking screw is formed of a first material; and
wherein the tissue manipulator is formed of a second material that is the same as the first material.

12. A tissue fixation system comprising:
a locking screw having a locking screw lengthwise axis, a locking screw first end, a locking screw second end, a locking screw axial length extending from the locking screw first end to the locking screw second end, and a locking screw main body defining a locking screw passageway extending from the locking screw first end to the locking screw second end, a locking screw exterior thread, a locking screw interior thread, and a recess, the locking screw exterior thread extending along a portion of the locking screw axial length and having a locking screw exterior thread first end and a locking screw exterior thread second end, the locking screw interior thread disposed within the locking screw passageway and extending along a portion of the locking screw axial length and having a locking screw interior thread first end and a locking screw interior thread second end, the locking screw exterior thread extending from the locking screw exterior thread first end to the locking screw exterior thread second end and around the locking screw lengthwise axis in a first direction, the locking screw interior thread extending from the locking screw interior thread first end to the locking screw interior thread second end and around the locking screw lengthwise axis in a second direction that is the same as the first direction, the recess extending from the locking screw first end toward the locking screw second end and in communication with the locking screw passageway; and
a tissue manipulator having a tissue manipulator first end, a tissue manipulator second end, a tissue manipulator axial length extending from the tissue manipulator first end to the tissue manipulator second end, a shaft, and a coil, the shaft partially disposed through the locking screw passageway and having a shaft lengthwise axis, a shaft first end, a shaft second end, a shaft exterior surface, and a shaft main body extending from the shaft first end to the shaft second end and defining a shaft exterior thread extending along a portion of the tissue manipulator axial length, the shaft exterior thread sized and configured to mate with the locking screw interior thread and having a shaft exterior thread first end and a shaft exterior thread second end, the shaft exterior thread extending from the shaft exterior thread first end to the shaft exterior thread second end and around the tissue manipulator lengthwise axis in a third direction that is the same as the first direction, the coil defining a plurality of turns and extending from the shaft second end to the tissue manipulator second end and around the tissue manipulator lengthwise axis in a fourth direction that is different than the first direction.

13. The tissue fixation system of claim 12, wherein the first direction is clockwise.

14. The tissue fixation system of claim 12, wherein the first direction is counterclockwise.

15. The tissue fixation system of claim 12, wherein the locking screw exterior thread first end is disposed between the locking screw first end and the locking screw second end;
wherein the locking screw exterior thread second end is disposed between the locking screw exterior thread first end and the locking screw second end.

16. The tissue fixation system of claim 12, wherein the shaft has a shaft outside diameter; and
wherein the coil has a coil outside diameter that is less than the shaft outside diameter.

17. The tissue fixation system of claim 12, wherein the shaft has a shaft first portion, and a shaft second portion, the shaft first portion extending from the shaft first end toward the shaft second end and having a shaft first portion outside diameter, the shaft second portion extending from the shaft first portion to the shaft second end and having a shaft second portion outside diameter that is less than the shaft first portion outside diameter.

18. The tissue fixation system of claim 17, wherein the shaft first portion outside diameter tapers to the shaft second portion outside diameter along a portion of the tissue manipulator axial length.

19. The tissue fixation system of claim 12, wherein the shaft exterior thread has a shaft exterior thread pitch; and
wherein the coil has a coil pitch that is the same as the thread pitch.

20. A tissue fixation system comprising:
a locking screw having a locking screw lengthwise axis, a locking screw first end, a locking screw second end, a locking screw axial length extending from the locking screw first end to the locking screw second end, and a locking screw main body defining a locking screw passageway extending from the locking screw first end to the locking screw second end, a locking screw exterior thread, a locking screw interior thread, and a recess, the locking screw exterior thread extending along a portion of the locking screw axial length and having a locking screw exterior thread first end and a locking screw exterior thread second end, the locking screw interior thread disposed within the locking screw passageway and extending along a portion of the locking screw axial length and having a locking screw interior thread first end and a locking screw interior thread second end, the locking screw exterior thread extending from the locking screw exterior thread first end to the locking screw exterior thread second end and around the locking screw lengthwise axis in a counterclockwise direction, the locking screw interior thread extending from the locking screw interior thread first end to the locking screw interior thread second end and around the locking screw lengthwise axis in a second direction that is the same as the first direction, the recess extending from the locking screw first end toward the locking screw second end and in communication with the locking screw passageway; and
a tissue manipulator having a tissue manipulator first end, a tissue manipulator second end, a tissue manipulator axial length extending from the tissue manipulator first end to the tissue manipulator second end, a shaft, and a coil, the shaft partially disposed through the locking screw passageway and having a shaft lengthwise axis, a shaft first end, a shaft second end, a shaft exterior surface, and a shaft main body extending from the shaft first end to the shaft second end and defining a shaft exterior thread extending along a portion of the tissue manipulator axial length, the shaft exterior thread sized and configured to mate with the locking screw interior thread and having a shaft exterior thread first end, a shaft exterior thread second end, and a shaft exterior thread pitch, the shaft exterior thread extending from the shaft exterior thread first end to the shaft exterior thread second end and around the tissue manipulator lengthwise axis in a third direction that is the same as the first direction, the coil defining a plurality of turns and extending from the shaft second end to the tissue manipulator second end and around the tissue manipulator lengthwise axis in a fourth direction that is different than the first direction, the coil having a coil pitch that is the same as the shaft exterior thread pitch.
